(19) United States
(12) Patent Application Publication
(10) Pub. No.: US 2008/0027011 A1
(43) Pub. Date: Jan. 31, 2008

(54) Homogeneous paste and gel formulations

(76) Inventors: Hassan Nached, Cranbury, NJ (US); Keith Freehauf, Stockton, NJ (US); Peter Hanson, Suwanee, GA (US)

Correspondence Address: Judy Jarecki-Black, Ph.D.,J.D., 3239 Satellite Blvd., Duluth, GA, 30096, US

(21) Appl. No.: 11/713,881

(22) Filed: Mar. 5, 2007

Related U.S. Application Data
(63) Continuation-in-part of application No. PCT/US06/48672, filed on Dec. 19, 2006.
(63) Continuation-in-part of application No. 11/314,387, filed on Dec. 20, 2005.

(51) Int. Cl. A61K 31/341; A61K 31/18; A61K 31/351; A61K 31/415; A61K 31/42; A61K 31/421; A61K 31/44; A61K 31/501; A61K 31/7048; A61K 47/02; A61P 31/00

(52) U.S. Cl. 514/28; 514/252.05; 514/341; 514/374; 514/378; 514/406; 514/456; 514/473; 514/605; 514/769

(57) ABSTRACT

This invention provides for a pharmaceutical or veterinary paste or gel formulation which may comprise an effective amount of a therapeutic agent; optionally an absorbent or a viscosity modifier; optionally a hydrophilic carrier, a colorant, stabilizer, surfactant, or preservative and optionally an antioxidant, solvent, flavoring, buffering system or thickener and methods of preparing these formulations. This invention also provides for, inter alia, oral homogeneous veterinary pastes or gels for the treating, controlling and preventing of endo- and ectoparasite infections in warm-blooded animals, such as birds, horses and household pets.

### INCORPORATION BY REFERENCE

This application is a continuation-in-part of international patent application Serial No. PCT/US06/48672 filed Dec. 19, 2006, which claims priority to U.S. patent application Ser. No. 11/314,387 filed Dec. 20, 2005, which published as U.S. patent publication No. 20060205681 on Sep. 14, 2006, which is a continuation-in-part application of U.S. patent application Ser. No. 11/107,048 filed Apr. 15, 2005, which is a divisional of U.S. patent application Ser. No. 10/177,822 filed Jun. 21, 2002, now U.S. Pat. No. 7,001,889 issued Feb. 21, 2006.

This application is also a continuation in part of U.S. patent application Ser. No. 11/314,387 filed Dec. 20, 2005, which published as U.S. patent publication No. 20060205681 on Sep. 14, 2006, which is a continuation-in-part application of U.S. patent application Ser. No. 11/107,048 filed Apr. 15, 2005, which is a divisional of U.S. patent application Ser. No. 10/177,822 filed Jun. 21, 2002, now U.S. Pat. No. 7,001,889 issued Feb. 21, 2006.

Reference is also made to U.S. Pat. Nos. 6,787,342 issued Sep. 7, 2004 and 6,939,881 issued Sep. 6, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention provides for oral homogeneous veterinary pastes and gels which are used in treating, controlling and preventing of endo- and ectoparasite infections in warm-blooded animals, such as birds, horses and household pets. This invention further provides for a process of preparing these veterinary pastes and gels and for a method for increasing the bioavailability of the agents contained in the paste or gel in the warm-blooded animal.

### BACKGROUND OF THE INVENTION

Therapeutic agents are administered to animals by a variety of routes. These routes include, for example, oral ingestion, topical application or parental administration. The particular route selected by the practitioner depends upon factors such as the physiochemical properties of the pharmaceutical or therapeutic agent, the condition of the host, and economic factors.

For example, one method of formulating a therapeutic agent for oral, topical, dermal or subdermal administration is to formulate the therapeutic agent as a paste or as an injectable formulation and reference is made to U.S. application Ser. No. 09/504,741, filed Feb. 16, 2000, now U.S. Pat. No. 6,787,342, entitled IMPROVED PASTE FORMULATIONS or to Ser. No. 09/346,905, filed Jul. 2, 1999, now U.S. Pat. No. 6,239,112; Ser. No. 09/112,690, filed Jul. 9, 1999 now U.S. Pat. No. 5,958,888 and Ser. No. 09/152,775, filed Sep. 14, 1998, now U.S. Pat. No. 6,174,540, entitled LONG ACTING INJECTABLE FORMULATIONS CONTAINING HYDROGENATED CASTOR OIL. The disclosure of these patent applications as well as the references cited therein and the references cited herein as well as the references cited in the references are expressly incorporated by reference. Other methods include placing the therapeutic agent in a solid or liquid matrix for oral delivery.

An important area in veterinary science in the control of endo- and ectoparasites in warm-blooded animals, such as equine animals and domestic pets. Infections of parasites, including cestodes and nematodes, commonly occur in animals such as horse, donkeys, mules, zebras, dogs, cats. Various classes anthelmintic agents have been developed in the art to control these infections; see, e.g., U.S. Pat. Nos. 3,993,682 and 4,032,655, which disclose phenylguanidines as anthelmintic agents. Further, the art recognizes that it is advantageous to administer combinations of two or more different classes of anthelmintic agents in order to improve the spectrum of activity; see, e.g., product disclosure for RM® Parasiticide-10, an anthelmintic paste comprising febantel and praziquantel.

Macrolide anthelmintic compounds are known in the art for treating endo- and ectoparasite infections in warm-blooded animals. Compounds that belong to this class of agents include the avermectin and milbemycin series of compounds. These compounds are potent antiparasitic agents against a wide range of internal and external parasites. Avermectins and milbemycins share the same common 16-membered macrocyclic lactone ring; however, milbemycins do not possess the disaccharide substituent on the 13-position of the lactone ring. In addition to treating parasitic insects, such as flies, avermectins and milbemycins are used to treat endoparasites, e.g., round worm infections, in warm-blooded animals.

The avermectin and milbemycin series of compounds either are natural products or are semi-synthetic derivatives. The natural product avermectins are disclosed in U.S. Pat. No. 4,310,519 to Albers-Schonberg, et al., and the 22,23-dihydro avermectin compounds are disclosed in Chabala, et al., U.S. Pat. No. 4,199,569. For a general discussion of avermectins, which include a discussion of their uses in humans and animals, see “Ivermectin and Abamectin,” W. C. Campbell, ed., Springer-Verlag, New York (1989). Naturally occurring milbemycins are described in Aoki et al., U.S. Pat. No. 3,950,360 as well as in the various references cited in “The Merck Index” 12^{th} ed., S. Budavari, Ed., Merck & Co., Inc. Whitehouse Station, N.J. (1996). Semisynthetic derivatives of these classes of compounds are well known in the art and are described, for example, in U.S. Pat. No. 5,077,308, U.S. Pat. No. 4,859,657, U.S. Pat. No. 4,963,582, U.S. Pat. No. 4,855,317, U.S. Pat. No. 4,871,719, U.S. Pat. No. 4,874,749, U.S. Pat. No. 4,427,663, U.S. Pat. No. 4,310,519, U.S. Pat. No. 4,199,569, U.S. Pat. No. 5,055,596, U.S. Pat. No. 4,973,711, U.S. Pat. No. 4,978,677, and U.S. Pat. No. 4,920,148. All these documents are herein incorporated by reference.

Avermectins and milbemycins are ineffective against cestodes, such as tapeworms, which also are a common parasite in warm-blooded animals (see, U.S. Pat. No. 6,207,179). Of particular importance in the industry is the treatment of equine tapeworms, in general, and Anoplacephala perfoliata, in particular (see, e.g., U.S. Pat. No. 6,207,179 or U.S. Pat. No. 5,824,653). In order to treat cestode (and trematode) infections in warm-blooded animals, it is know, to administer 2-acyl-4-oxo-pyrazino-isoquinoline derivatives to the animal (see, e.g., U.S. Pat. No. 4,001,441, herein incorporated by reference). A compound of this class that is often used to treat cestode and nematode infections is praziquantel, which has the following structure:

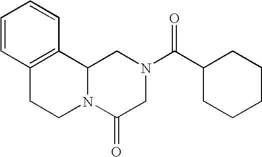

As mentioned above, often it is beneficial to administer a formulation that contains a combination of two or more anthelmintics, which possess different activity, in order to obtain a composition with a broad spectrum of activity. Further, the combination allows the user to administer one formulation instead of two or more different formulations to the animal. Formulations which administer a combination of two or more anthelmintics are know in the art. These formulations may be in the form of solutions, suspensions, pastes, drenches or pour-on formulations (see, e.g., U.S. Pat. No. 6,165,987 to Harvey or U.S. Pat. No. 6,340,672 to Mihalik). For example, U.S. Pat. No. 4,468,390 to Kitano and U.S. Pat. No. 5,824,653 to Beuvry et al. describe anthelmintic compositions for treating nematode and cestode infections in animals, such as horses, that comprise an avermectin or a milbemycin and an isoquinoline compounds, such as praziquantel, to the animal. In these formulations, the avermectin or milbemycin compound and the isoquinoline compound are not dissolved in a solvent, which is then dispersed in a semi-solid matrix. Similarly, U.S. Pat. No. 6,207,179 to Mihalik relates to anthelmintic paste formulations wherein the avermectin or milbemycin is dissolved in a non-aqueous liquid and pyrantel or morantel, compounds which are in the same class as praziquantel, but are said in the art to be far less effective as praziquantel, are suspended in the liquid. These prior patents do not describe a formulation wherein the both the praziquantel and the avermectin or milbemycin are dissolved in a solvent and then dispersed in a carrier matrix. U.S. Pat. No. 6,165,987 relates to anthelmintic formulations containing praziquantel and at least one avermectin or milbemycin dissolved in glycerol formal, benzyl alcohol and N-methyl-2-pyrrolidone, which may be liquids, pastes or drenches; the amount of praziquantel administered to the animal is always at a dose of more that 2.0 mg per kg of body weight. U.S. Pat. No. 6,165,987 provides for pastes which require the presence of two solvents, one for the praziquantel and one for the macrolide compound.

Citation or identification of any document in this application is not an admission that such document is available as prior art to the present invention.

### SUMMARY OF THE INVENTION

The present invention provides for a stable paste or gel formulation for a wide range of veterinary and pharmaceutical products. The present invention also provides for an improved process to make the inventive paste and gel products.

This invention provides for oral homogeneous veterinary pastes and gels for the treating, controlling and preventing of endo- and ectoparasite infections in warm-blooded animals, such as birds, horses and household pets, as well as to a process for preparing these formulations. The inventive oral veterinary pastes and gels provide for a more effective treatment of parasitic infections in non-human animals since the active ingredients do not interfere with each other, hence increasing the bioavailability in the animal, while still having the benefits of being administered by as a paste or a gel. Further, the inventive formulations provide for a formulation that exhibits good chemical and physical stability over the shelf-life of the product. Thus, the oral veterinary formulations of the invention may exhibit the benefits of both a solution and a formulation that is a paste or a gel.

In advantageous embodiment, the pharmaceutical or veterinary paste formulation of the present invention may comprise:

(a) an effective amount of a therapeutic agent, wherein the therapeutic agent is a COX-2 inhibitor,

(b) a colorant, wherein the colorant is titanium dioxide,

(c) an absorbent, wherein the absorbent is magnesium carbonate,

(d) a thickener, wherein the thickener is colloidal anhydrous silica and

(e) a viscosity modifier, wherein the viscosity modifier is PEG 300.

In another advantageous embodiment, the pharmaceutical or veterinary gel formulation of the present invention may comprise:

(a) an effective amount of a therapeutic agent,

(b) an antioxidant, wherein the antioxidant is sodium ascorbate,

(c) a preservative, wherein the preservative is potassium sorbate,

(d) a flavoring, wherein the flavoring is a peppermint and/or apple flavor,

(e) a buffering system or thickener, wherein the buffering system or thickener is sodium carboxymethylcellulose,

(f) optionally a solvent, wherein the solvent is propylene glycol and

(g) optionally a viscosity modifier, wherein the viscosity modifier is PEG 400.

Advantageously, the therapeutic agent may comprise an avermectin, advantageously eprinomectin or ivermectin. In other advantageous embodiments, the therapeutic agent may comprise a COX-2 inhibitor, a proton pump inhibitor, advantageously omeprazole, praziquantel or any combination thereof, including one or more avermectins.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 depicts leverage plots of variables and whole model and

FIG. 2 depicts a prediction profiler.

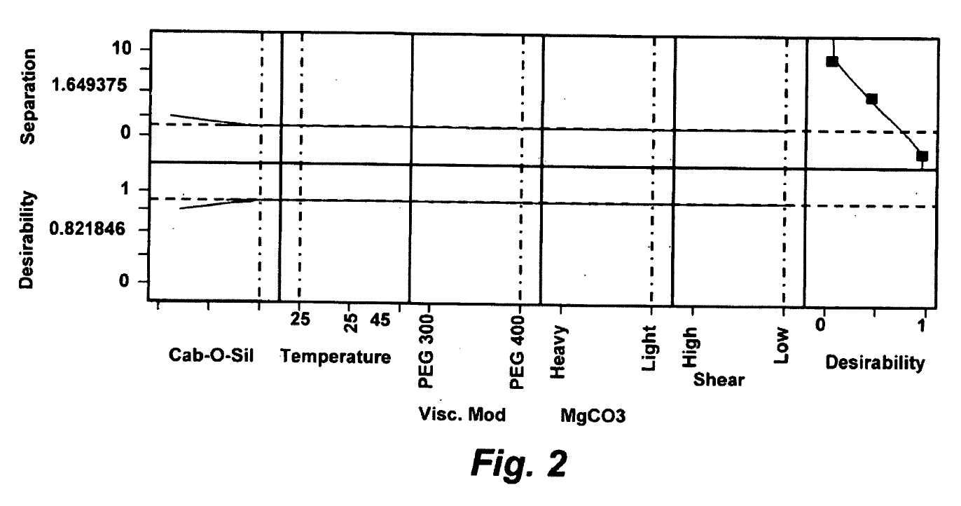

### DETAILED DESCRIPTION

The present invention provides for a stable paste or gel formulation for a wide range of veterinary and pharmaceutical products. The present invention also provides for an improved process to make the inventive paste or gel products. In particular, the paste or gel formulation of the present invention provides for lower percentage of active ingredient with a resulting increase in the percentage of solvent,

The present invention provides for a pharmaceutical or veterinary paste or gel formulation comprising:

(a) an effective amount of a therapeutic agent;

(b) optionally, a fumed silica;

(c) a viscosity modifier;

(d) a carrier;

(e) optionally, an absorbent; and

(f) optionally, a colorant, stabilizer, surfactant, or preservative.

This invention also provides for a process for preparing a paste or gel formulation comprising the steps of:

(a) dissolving or dispersing the therapeutic agent into the carrier by mixing;

(b) adding the optional fumed silica and absorbent to the carrier containing the dissolved therapeutic agent, mixing at low shear (advantageously 300 rpm) and maintaining the temperature at about 25 C until the silica and absorbent is dispersed in the carrier and

(c) adding the viscosity modifier to the intermediate with mixing to produce a uniform paste or gel.

The steps are illustrating, but not limiting. For example, step (a) can be moved to the last step.

More preferred are pharmaceutical and veterinary pastes or gels comprising:

(a) a therapeutic agent selected from the group consisting of insecticides, acaricides, parasiticides, growth enhancers, oil-soluble NSAIDS or a proton pump inhibitor;

(b) optionally, a fumed silica;

(c) a viscosity modifier;

(d) an absorbent;

(e) a colorant; and

(f) a carrier which is triacetin, a monoglyceride, a diglyceride, or a triglyceride.

Also preferred are pastes or gels comprising:

(a) a therapeutic agent selected from the group consisting of avermectins, milbemycins, nordulisporic acid and its derivatives, estrogens, progestins, androgens, substituted pyridyl methyl derivatives, phenylpyrazoles, COX-2 inhibitors or 2-(2-benzimidazolyl)-pyrimidine derivatives;

(b) optionally, a fumed silica;

(c) a viscosity modifier;

(d) an absorbent;

(e) a colorant; and

(f) a hydrophilic carrier which is triacetin, a monoglyceride, a diglyceride, or a triglyceride.

The above compositions wherein the fumed silica is a colloidal silicon dioxide such as CAB-O-SIL (Cabot, TD11) or AEROSIL (Degussa, Technical Bulletin Pigments, No. 11 and No. 49), the viscosity modifier is a polyethylene glycol such as PEG 200, PEG 300, PEG 400, PEG 600, monoethanolamine, triethanolamine, glycerol, propylene glycol, polyoxyethylene (20) sorbitan mono-oleate (polysorbate 80 or Tween 80), polyoxamers (e.g., Pluronic L 81); the absorbent is magnesium carbonate, calcium carbonate, starch, or cellulose and its derivatives; and the colorant is titanium dioxide iron oxide, or FD&C Blue #1 Aluminum Lake are most especially preferred.

In an advantageous embodiment, the fumed silica is a colloidal silicon dioxide such as CAB-O-SIL (Cabot, TD11), advantageously at a concentration of 5% w/w, the viscosity modifier is PEG 400 and the absorbent is magnesium carbonate.

In an advantageous embodiment, the viscosity modifier is PEG 300. Advantageously, the PEG 300 is at a concentration of about 0.4% w/w.

Advantageously, the absorbent is magnesium carbonate, in particular a heavy magnesium carbonate. In an advantageous embodiment, the magnesium carbonate is at a concentration of about 2% w/w.

In another advantageous embodiment, the colorant is titanium dioxide, in particular E171 titanium dioxide. Advantageously, the titanium dioxide is at concentration of about 0.2% w/w.

Advantageously, the carrier is triacetin. In an advantageous embodiment, the triacetin is at a concentration of up to about 100% w/w depending on the volume of the active substance and other excipients.

The therapeutic agents which are used in the inventive formulations are those which are known to the practitioner as agents which may be formulated as pastes or gels. Classes of therapeutic agents contemplated by the inventive formulations include insecticides, acaricides, parasiticides, growth enhancers, oil-soluble, nonsteroidal anti-inflammatory drugs (NSAIDS), proton pump inhibitors and antibacterial compounds. Specific classes of compounds which fall within these classes include, for example, avermectins, milbemycins, nodulisporic acid and its derivatives, estrogens, progestins, androgens, substituted pyridylmethyl derivatives, phenylpyrazoles, COX-2 inhibitors, 2-(2-benzimidazolyl)-pyrimidines derivatives, depsipeptides (such as emodepside) and macrolide antibiotics.

In an advantageous embodiment, the therapeutic agent is a non-steroidal anti-inflammatory drug (NSAID), in particular a COX-2 inhibitor. In a particularly advantageous embodiment, the COX-2 inhibitor is firocoxib. In a most advantageous embodiment, the firocoxib is at a concentration of about 0.82% w/w.

Firocoxib is a veterinary COX-2 inhibitor (anti-inflammatory and anti-arthritic) with a primary application in canines. Exemplary patents include, but are not limited to, U.S. Pat. No. 6,677,373, which covers polymorphic Form B of 3-(cyclopropylmethoxy)-4-[4-(methylsulfonyl)phenyl]-5,5-dimethyl-5H-furan-2-one and U.S. Pat. No. 5,981,576, which covers the compound 3-(cyclopropylmethoxy)-4-[4-(methylsulfonyl)phenyl]-5,5-dimethyl-5H-furan-2-one. See also, e.g., AU0079218A5, AU0774172B2, AU0774172C, AU4206979AA, CA2386549AA, EP1090915A1, JP2003511443T2, US20030050337A1, U.S. Pat. No. 6,677,373 and WO0127097A1 and AT0304537E, AU0077944A5, AU0773221B2, CA2386807AA, DE60022663C0, DE60022663T2, DK1218366T3, EP1218366A2, EP1218366B1, EP1218366B8, ES2249299T3, FR2799462A1, FR2799462B1, JP2003511444T2, US20030028036A1, U.S. Pat. No. 6,541,646, U.S. Pat. No. 6,541,646B2, WO0127098A2 and WO0127098A3.

The present invention further encompasses other COX-2 inhibitors in addition to firocoxib.

The term “cyclooxygenase-2 inhibitor” means any pharmaceutically acceptable compound or combination of compounds, including salts, tautomers and prodrugs of such compound or compounds, that inhibits the enzyme cyclooxygenase-2 in the arachidonic acid/prostaglandin pathway. The specific cyclooxygenase-2 inhibitor or inhibitors used in the food composition-are not narrowly critical so long as the inhibitor or inhibitors are pharmaceutically acceptable and are compatible with the specific processing conditions selected.

The cyclooxygenase-2 inhibitors employed in this invention include, but are not limited to, the compounds corresponding to the structural formula:

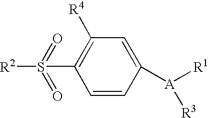

wherein A is a 5- or 6-member ring substituent selected from partially unsaturated or unsaturated heterocyclo and carbocyclic rings;

wherein R¹ is cyclohexyl or phenyl optionally substituted with one, two or three radicals selected from C₁₋₂ alkyl, C₁₋₂ haloalkyl, cyano, carboxyl, C₁₋₂ alkoxycarbonyl, hydroxyl, C₁₋₂ hydroxyalkyl, C₁₋₂ haloalkoxy, amino, C₁₋₂ alkylamino, phenylamino, nitro, C₁₋₂ alkoxy-C₁₋₂ alkyl, C₁₋₂ alkylsulfinyl, halo, C₁₋₂ alkoxy and C₁₋₂ alkylthio;

wherein R² is methyl or amino;

wherein R³ is a radical selected from halo, C₁₋₂ alkyl, alkenyl, alkynyl, oxo, cyano, carboxyl, cyanoalkyl, heterocyclyloxy, C₁₋₂ alkyloxy, alkylthio, alkylcarbonyl, cycloalkyl, phenyl, C₁₋₂ haloalkyl, heterocyclo, cycloalkenyl, phenylalkyl, heterocyclylalkyl, alkylthioalkyl, C₁₋₂ hydroxyalkyl, alkoxycarbonyl, phenylcarbonyl, phenylalkylcarbonyl, phenylalkenyl, alkoxyalkyl, phenylylthioalkyl, phenylyloxyalkyl, phenylalkylthioalkyl, phenylalkoxyalkyl, alkoxyphenylalkoxyalkyl, alkoxycarbonylalkyl, aminocarbonyl, aminocarbonylalkyl, alkylaminocarbonyl, N-phenylaminocarbonyl, N-alkyl-N-phenylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-phenylaminoalkyl, N-phenylalkylaminoalkyl, N-alkyl-N-phenylalkylaminoalkyl, N-alkyl-N-phenylaminoalkyl, phenyloxy, phenylalkoxy, phenylthio, phenylalkylthio, alkylsulfinyl, alkylsulfonyl, aminosulfonyl, alkylaminosulfonyl, N-phenylaminosulfonyl, phenylsulfonyl and N-alkyl-N-phenylaminosulfonyl; and

wherein R⁴ is hydrido or fluoro;

or a pharmaceutically-acceptable salt thereof.

A class of cyclooxygenase-2 inhibitors of particular interest consists of those compounds with the structural formula:

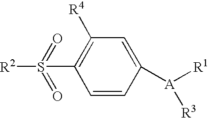

wherein A is a 5- or 6-member ring substituent selected from partially unsaturated or unsaturated heterocyclo and carbocyclic rings selected from the group consisting of thienyl, oxazolyl, furyl, pyrrolyl, thiazolyl, imidazolyl, benzofuryl, indenyl, benzothienyl, isoxazolyl, pyrazolyl, cyclopentenyl, cyclopentadienyl, benzindazolyl, benzopyranopyrazolyl, phenyl, and pyridyl; wherein R¹ is cyclohexyl or phenyl optionally substituted with one, two or three radicals selected from C₁₋₂ alkyl, halo, and C₁₋₂ alkoxy; wherein R² is methyl or amino; wherein R³ is a radical selected from halo, C₁₋₂ alkyl, oxo, cyano, carboxyl, C₁₋₂ alkyloxy, phenyl, C₁₋₂ haloalkyl, and C₁₋₂ hydroxyalkyl; and wherein R⁴ is hydrido or fluoro; or a pharmaceutically-acceptable salt thereof.

In another advantageous embodiment, the COX-2 inhibitor is 3-(cyclopropylmethoxy)-5,5-dimethyl-4-[4-(methylsulfonyl)phenyl]-2(5H)-furanone with a CAS number of 189954-96-9 which has the structure:

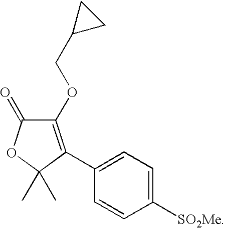

Other COX-2 inhibitors that may be employed in the present invention include, but are not limited to, lomoxicam, 1,5-diphenyl-3-substituted pyrazoles, radicicol, N-benzyl-3-indoleacetic acids, the inhibitors of GB-02283745, TP-72, indene inhibitors of COX-2, carbocyclic diarylmethylene derivatives, 1,2-diarylindole, 1,2-bisarylcyclobutene derivatives, novel stilbene derivatives as prodrug forms of the diphenylcyclopentenonees of U.S. Pat. No. 5,474,995, WO-09500501 and WO-09518799, 2,4-diphenylbutenoic acid derivatives as prodrugs of COX-2 inhibitors claimed in U.S. Pat. No. 5,474,995, WO-09500501 and WO-09518799, 1-(4-chlorobenzoyl)-3-[4-(4-fluorophenyl)thiazol-2-ylmethyl]-5-methoxy-2-methylindole, sulfonamide substituted diarylthiazole, 4-(4-cyclohexyl-2-methyloxazol-5-yl)-2-fluorobenzenesulfonamide, 5,6-diarylthiazolo[3,2-B][1,2,4]triazolo, indometacin-derived indolalkanoic acid, 1-methylsulfonyl-4-[1,1-dimethyl-4-(4-fluorophenyl)cyclopenta-2,4-dien-3-yl]benzene, 4,4-dimethyl-2-phenyl-3-[4-(methylsulfonyl)phenyl]cyclobutenone; 1,2-diarylcyclobutenes, 2-(4-methoxyphenyl)-4-methyl-1-(4-sulfamoylphenyl)pyrrole; 1,2-diphenylpyrrole derivatives, tetrahydrofuranones, N-[5-(4-fluoro)phenoxyl]thiophene-2-methanesulfonamide, 5(E)-(3,5-di-tert-butyl-4-hydroxy)benzylidene-2-ethyl-1,2-isothiazolidine-1,1-dioxide, 3-formylamino-7-methylsulfonylamino-6-phenoxy-4H-1-benzopyran-4-one, benzenesulfonamide, 4-(5-(4-methylphenyl)-3-(trifluoromethyl)-1H-pyrazole-1-yl)-, 2H-1,2-benzothiazine-3-carboxamide, 4-hydroxy-2-methyl-N-(5-methyl-2-thiazolyl)-, 1,1-dioxide-, methanesulfonamide, N-(4-nitro-2-phenoxyphenyl) and methanesulfonamide, N-(4-nitro-2-phenoxyphentyl).

Other COX-2 inhibitors that may be employed in the present invention include, but are not limited to, the COX-2 inhibitors disclosed in AT 9700165, AU 9719132, CA 2164559, DE 19518421, DE 19533643, DE 19533644, DE 19753463, EP 714895, EP 799823, EP 832652, EP 846689, EP 850894, EP 850895, EP 95909447.5, EP 95928164.3, FR 2751966, GB 2283745, GB 2294879, GB 2319772, GB 2320715, JP 08157361, JP 09048769, JP 09071656, JP 09071657, JP 09077664, JP 09194354, JP 09221422, JP 10175861, PCT Application US97/05497, PCT Application US98/07677, SOFRC 95/1107, U.S. Pat. No. 5,380,738, U.S. Pat. No. 5,380,738, U.S. Pat. No. 5,393,790, U.S. Pat. No. 5,418,254, U.S. Pat. No. 5,420,343, U.S. Pat. No. 5,434,178, U.S. Pat. No. 5,466,823, U.S. Pat. No. 5,474,995, U.S. Pat. No. 5,486,534, U.S. Pat. No. 5,504,215, U.S. Pat. No. 5,508,426, U.S. Pat. No. 5,510,368, U.S. Pat. No. 5,510,496, U.S. Pat. No. 5,516,907, U.S. Pat. No. 5,521,207, U.S. Pat. No. 5,547,975, U.S. Pat. No. 5,563,165, U.S. Pat. No. 5,565,482, U.S. Pat. No. 5,576,339, U.S. Pat. No. 5,580,985, U.S. Pat. No. 5,596,008, U.S. Pat. No. 5,604,260, U.S. Pat. No. 5,616,458, U.S. Pat. No. 5,616,601, U.S. Pat. No. 5,620,999, U.S. Pat. No. 5,633,272, U.S. Pat. No. 5,643,933, U.S. Pat. No. 5,663,180, U.S. Pat. No. 5,663,195, U.S. Pat. No. 5,668,161, U.S. Pat. No. 5,670,510, U.S. Pat. No. 5,670,532, U.S. Pat. No. 5,672,626, U.S. Pat. No. 5,677,318, U.S. Pat. No. 5,677,318, U.S. Pat. No. 5,681,842, U.S. Pat. No. 5,686,460, U.S. Pat. No. 5,686,470, U.S. Pat. No. 5,696,143, U.S. Pat. No. 5,719,163, U.S. Pat. No. 5,719,163, U.S. Pat. No. 5,733,909, U.S. Pat. No. 5,736,579, U.S. Pat. No. 5,739,166, U.S. Pat. No. 5,753,688, U.S. Pat. No. 5,753,688, U.S. Pat. No. 5,756,530, U.S. Pat. No. 5,760,068, U.S. Pat. No. 5,776,967, U.S. Pat. No. 5,783,597, U.S. application Ser. No. 08/004/822, U.S. application Ser. No. 08/237,739, U.S. application Ser. No. 08/387,680, U.S. application Ser. No. 08/424,979, U.S. application Ser. No. 08/425,022, U.S. application Ser. No. 08/425,029, U.S. application Ser. No. 08/457,902, U.S. application Ser. No. 08/464,722, U.S. application Ser. No. 08/540,522, U.S. application Ser. No. 08/541,850, U.S. application Ser. No. 08/647,911, U.S. application Ser. No. 08/702,417, U.S. application Ser. No. 08/765,865, U.S. application Ser. No. 08/765,865, U.S. application Ser. No. 08/776,090, U.S. application Ser. No. 08/776,358, U.S. application Ser. No. 08/801,768, U.S. application Ser. No. 08/809,318, U.S. application Ser. No. 08/809,475, U.S. application Ser. No. 08/822,528, U.S. application Ser. No. 08/849,069, U.S. application Ser. No. 08/894,102, U.S. application Ser. No. 08/894,124, U.S. application Ser. No. 08/908,554, U.S. application Ser. No. 08/945,840, U.S. application Ser. No. 08/952,156, U.S. application Ser. No. 08/952,661, U.S. application Ser. No. 08/957,345, U.S. application Ser. No. 08/969,953, U.S. application Ser. No. 08/987,356, U.S. application Ser. No. 08/992,327, U.S. application Ser. No. 09/005,610, U.S. application Ser. No. 09/062,537, U.S. application Ser. No. 09/101,493, U.S. Application 60/032,688, U.S. Application 60/044,485, WO 9413635, WO 9414977, WO 9420480, WO 9426731, WO 9427980, WO 9427980, WO 9500501, WO 9511883, WO 9515316, WO 9515318, WO 9521817, WO 9530652, WO 9603385, WO 9603385, WO 9603385, WO 9603387, WO 9603387, WO 9603388, WO 9603392, WO 9606840, WO 9608482, WO 960923, WO 9609293, WO 9609304, WO 9609304, WO 9609304, WO 9611676, WO 9612483, WO 9613483, WO 9616934, WO 9619462, WO 9619462, WO 9619463, WO 9619463, WO 9619469, WO 9621667, WO 9623786, WO 9624584, WO 9624585, WO 9624585, WO 9624604, WO 9625405, WO 9625405, WO 9625928, WO 9626921, WO 9631509, WO 9636617, WO 9636623, WO 9637467, WO 9637469, WO 9638418, WO 9638442, WO 9638442, WO 9639144, WO 9640143, WO 9641625, WO 9641626, WO 9641626, WO 9641645, WO 9703667, WO 9703953, WO 9709977, WO 9710840, WO 9711701, WO 9711701, WO 9711704, WO 9713755, WO 9713767, WO 9714679, WO 9714691, WO 9716435, WO 9725045, WO 9725046, WO 9725047, WO 9725048, WO 9727181, WO 9727181, WO 9728120, WO 9728121, WO 9729774, WO 9729775, WO 9729776, WO 9730030, WO 9731631, WO 9734882, WO 9736497, WO 9736863, WO 9737984, WO 9738686, WO 9738986, WO 9740012, WO 9744027, WO 9744028, WO 9745420, WO 9746524, WO 9746532, WO 9800416, WO 9803484, WO 9804527, WO 9805639, WO 9806708, WO 9806715, WO 9807425, WO 9807714, WO 9811080, WO 9815528, WO 9816227, WO 9816227, WO 9817292, WO 9821195, WO 9821195, WO 9822101, WO 9822101, WO 9822104, WO 9822442, WO 9822457, WO 9824782, WO 9825896 and ZA 9704806.

Still more advantageous COX-2 inhibitors are selected from the group consisting of:

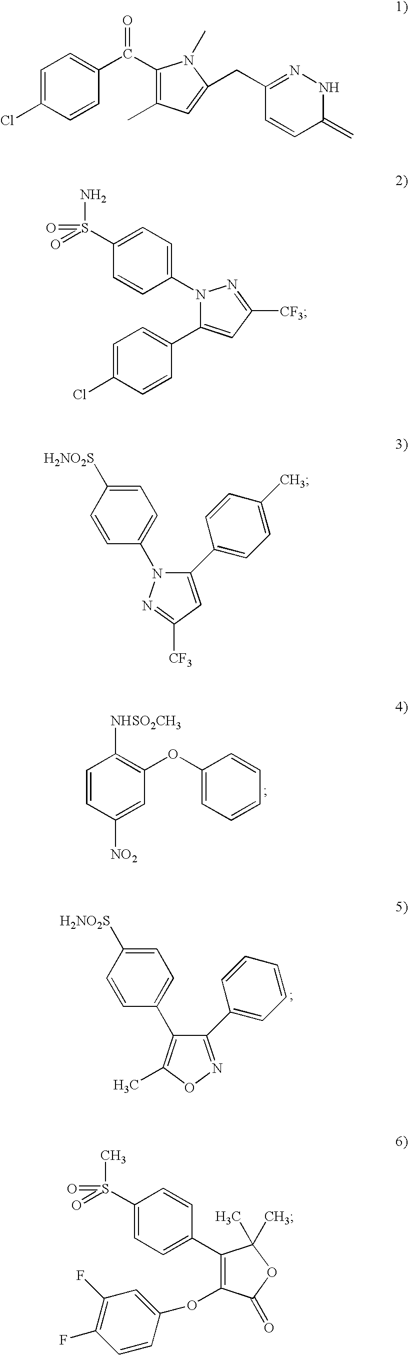

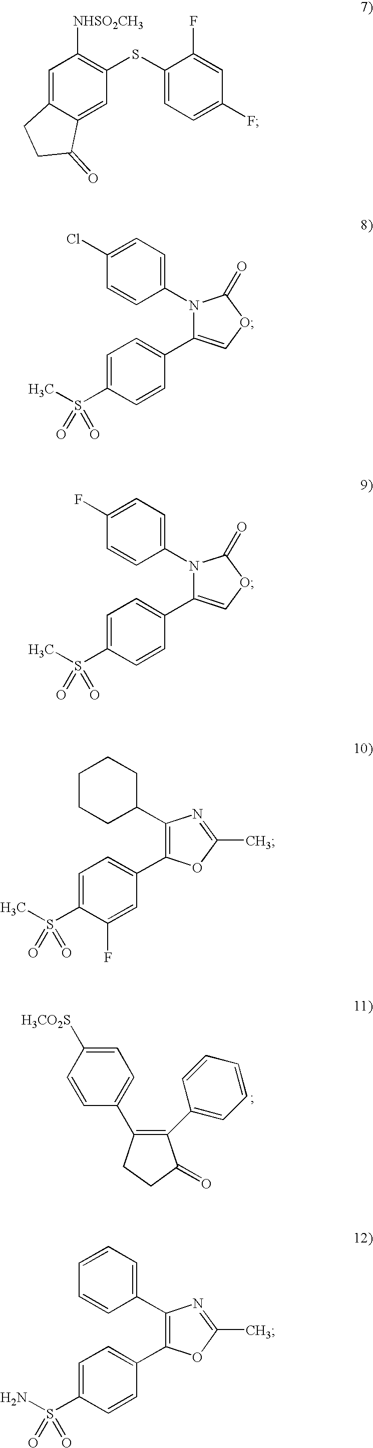

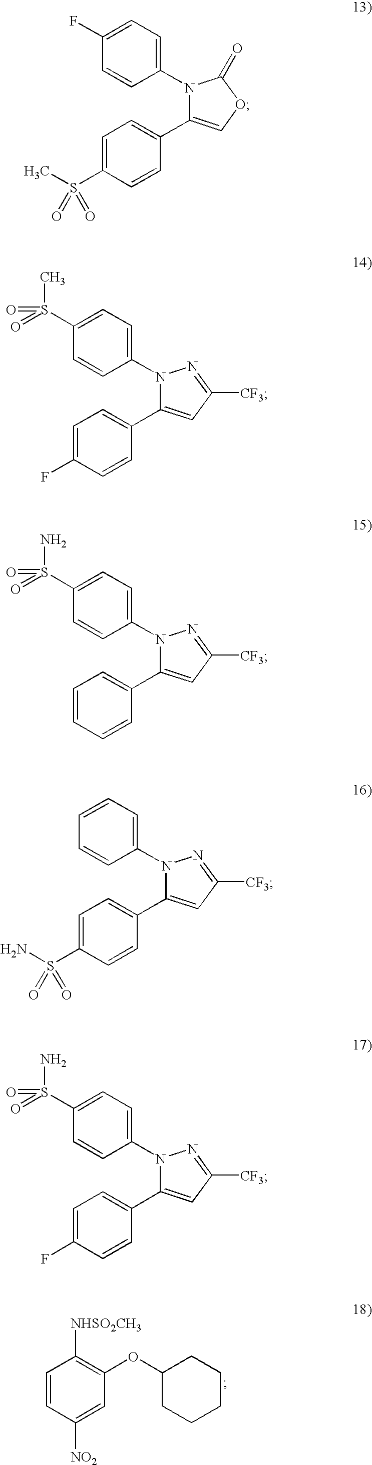

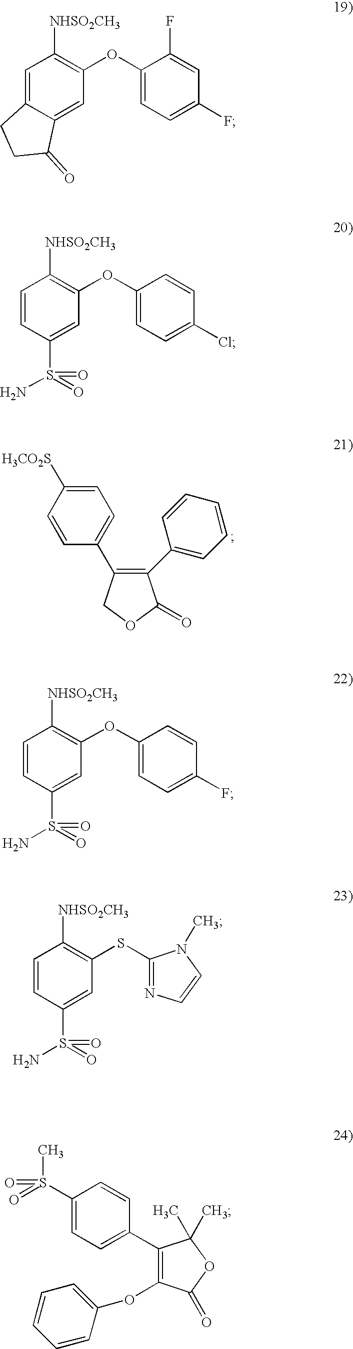

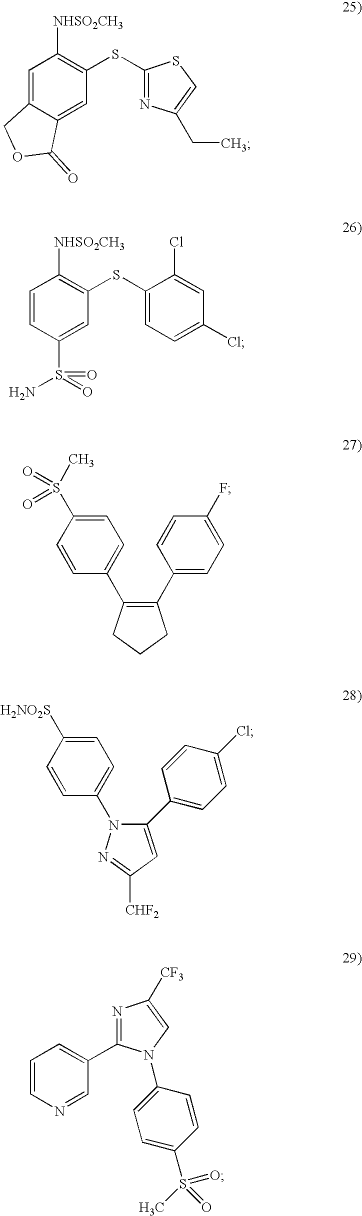

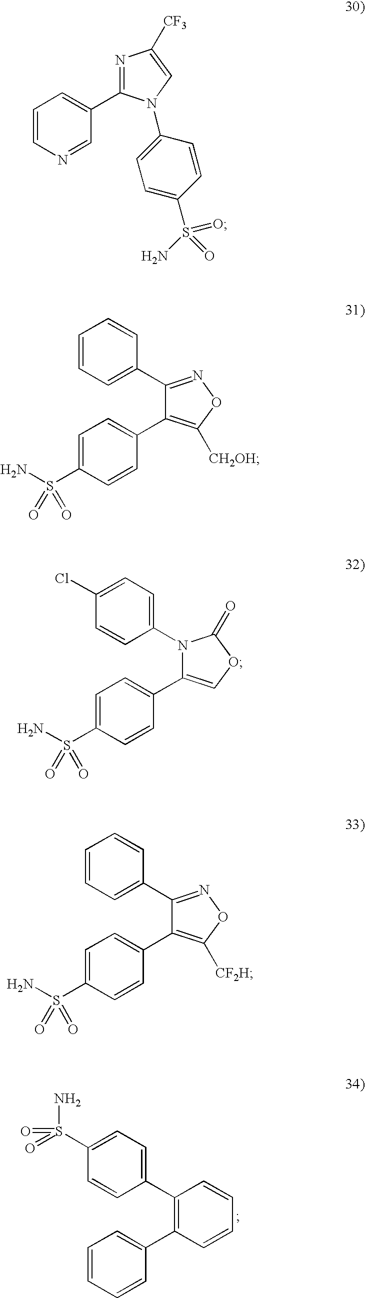

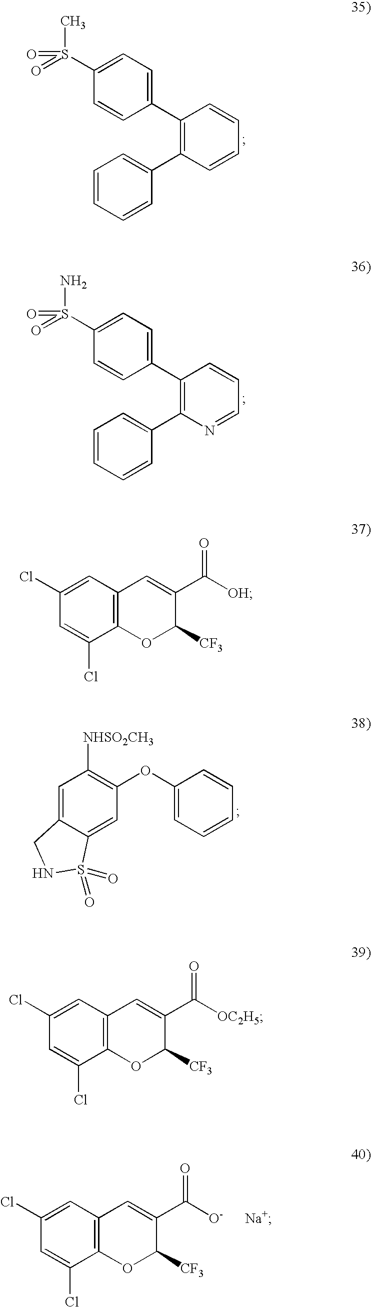

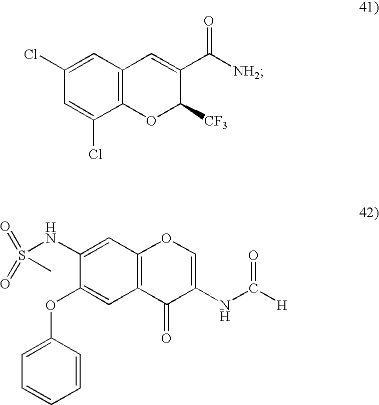

In another advantageous embodiment, the cyclooxygenase-2 inhibitors are selected from the group consisting of:

(1) the compound 4-[5-(4-methylphenyl)-3-(trifluoromethyl)-1H-pyrazole-1-yl]benzenesulfonamide which has the structure:

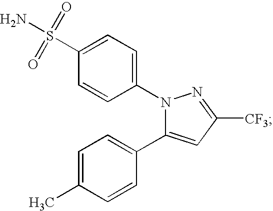

(2) the compound 4-[5-((3-fluoro-4-methoxy)phenyl)-3-(difluoromethyl)-1H pyrazole-1-yl]benzenesulfonamide which has the structure:

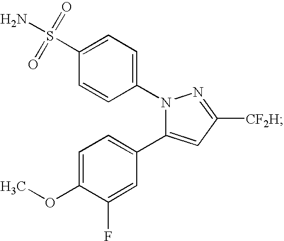

(3) the compound 4-[5-(4-chlorophenyl)-3-(trifluoromethyl)-1H-pyrazole-1-yl]benzenesulfonamide which has the structure:

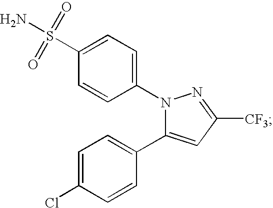

(4) the compound that has the structure:

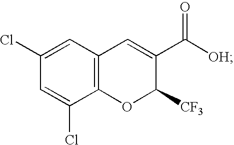

(5) the compound 4-[4-(methylsulfonyl)phenyl]-3-(4-(methoxy)phenyl)furan-2(5H)-one which has the structure:

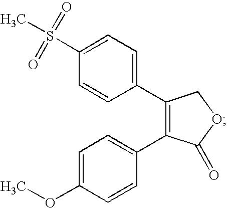

and (6) the compound 4-[5-methyl-3-phenylisoxazol-4-yl]benzenesulfonamide, which has the structure:

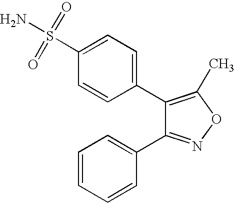

Compounds which inhibit gastric acid secretion in the stomach or act as proton pump inhibitors are well known to the practitioner and are also provided for in the present invention. These compounds include 2-(2-benzimidazolyl-pyridines) and their salts. Such compounds are described, for example in EP 005 129, U.S. Pat. No. 4,255,431 as well as in U.S. Pat. No. 5,629,305. These compounds are also known to treat Helicobacter infections, U.S. Pat. No. 5,093,342, and to act as synergists when combined with an acid degradable antibiotic, see e.g. U.S. Pat. No. 5,629,305. These synergistic combinations may also be formulated in the pastes of the present invention. Omeprazole or its salts is an especially preferred compound.

The proton pump inhibitors used in the present invention can include compounds of the general formula:

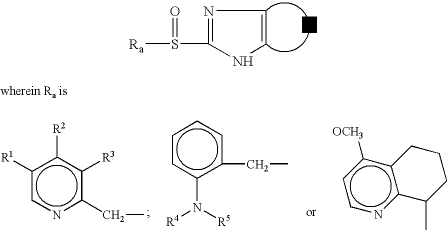

where R¹ and R³ are independently selected from hydrogen, lower alkyl, lower alkoxy and halogen, R² is selected from hydrogen, lower alkyl, lower alkoxy-lower alkoxy, lower fluoralkoxy and

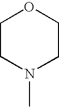

R⁴ and R⁵ are independently selected from lower alkyl,

A is

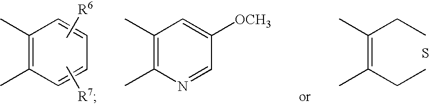

R⁶ and R⁷ are independently selected from hydrogen, lower alkyl, lower alkoxy, lower fluoroalkoxy, lower fluoroalkyl, halogen,

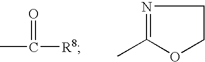

wherein R.sup.8 is lower alkyl or lower alkoxy.

Examples of proton pump inhibitors include:

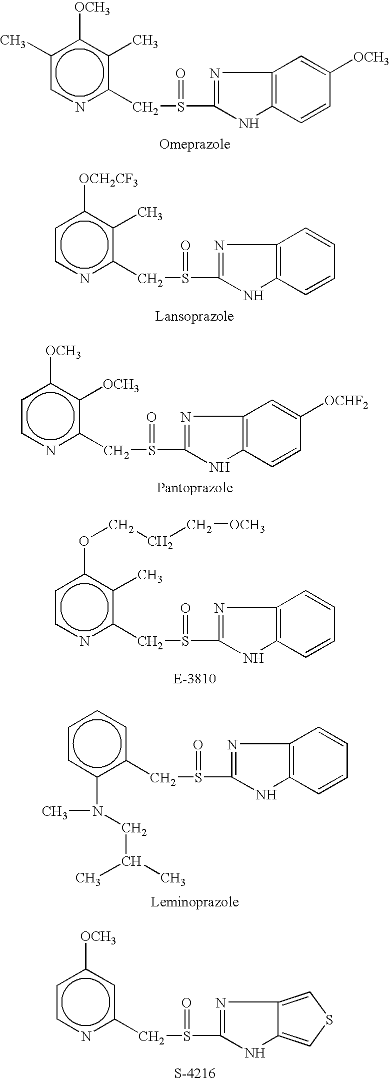

Examples of other PPI include esomeprazole (nee: perprazole), rabeprazole, and IY-81149 (distributed by Axican Pharma).

The preferred proton pump inhibitor used in the present invention is the compound known as omeprazole.

The proton pump inhibitors used in the present invention are known compounds in the art and methods for their preparation may be found in the literature. For example, omeprazole is disclosed in EP 5129, lansoprazole in EP 174.716, pantoprazole in EP 166,287, leminoprazole in GB 2,163,747, rabeprazole in U.S. Pat. No. 5,045,552.

The present invention also provides for an oral homogeneous anthelmintic veterinary paste or gel, for the treating, controlling and preventing of endo- and ectoparasite infections in warm-blooded animal, which comprises an anthelmintic agent, such a praziquantel, and/or pyrantel and, as a second agent, at least one macrolide anthelmintic agent, a solvent which dissolves both the first anthelmintic agent and the macrolide anthelmintic agent, and a thickening agent.

More specifically, this invention provides for an oral homogeneous veterinary paste or gel consisting essentially of praziquantel and/or pyrantel and at least one macrolide anthelmintic compound, a solvent, which dissolves both the praziquantel and/or pyrantel and the macrolide anthelmintic compound, and at least one thickening agent. Preferred are oral homogeneous veterinary pastes or gels consisting essentially of praziquantel and/or pyrantel and at least one macrolide anthelmintic compound, a solvent, which dissolves both the praziquantel and/or pyrantel and the macrolide anthelmintic compound, at least one thickening agent, and at least one viscosity modifier. Another embodiment of the invention is an oral veterinary composition consisting essentially of the inventive oral homogeneous veterinary pastes or gels and an opacifier.

The inventive oral homogeneous veterinary pastes or gels provide for the combination of at least two different anthelmintic agents, one of which is a macrolide anthelmintic compound. The classes of compounds encompassed by the first agent are well known to practitioners in this art. These compounds include, in addition to praziquantel and its related compounds, anthelmintic agents such as pyrantel (see, U.S. Pat. No. 3,502,661 for a description of pyrantel and its related compounds).

The invention provides for an oral homogeneous veterinary paste or gel consisting essentially of praziquantel and/or pyrantel and at least one macrolide anthelmintic compound, a solvent, which dissolves both the praziquantel and/or pyrantel and the macrolide anthelmintic compound, at least one thickening agent, and at least one viscosity modifier. In a preferred embodiment, the macrolide anthelmintic compound is selected from the group consisting of doramectin, abamectin, moxidectin, selamectin and ivermectin; the solvent is glycerol formal, propylene glycol, n-methylpyrrolidone, or dimethyl sulfoxide; the thickening agent is selected from the group consisting of a cellulose, a starch, monothioglycerol, polymers or copolymers of polyvinylpyrrolidone, polymers and copolymers of (meth)acrylate, and a natural gum; and the viscosity modifier is selected from the group consisting of vegetable oils, or hydrogenated vegetable oils. In a preferred embodiment, the thickening agent is hydroxypropylcellulose, xanthum gum or hydroxyethyl starch and the viscosity modifier is hydrogenated castor oil, corn oil or olive oil.

The macrolide anthelmintic compounds contemplated in this invention are also well known to a practitioner of this area. These compounds include avermectins and milbemycins, some of which are discussed above. Non-limiting examples of compounds belonging to this class are represented by the following structure: 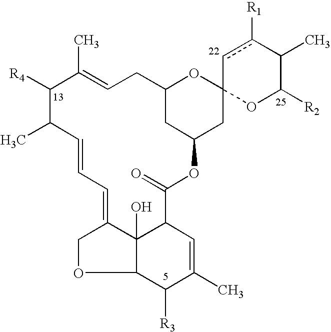
where the broken line indicates a single or a double bond at the 22,23-positions;

R₁ is hydrogen or hydroxy provided that R₁ is present only when the broken line indicates a single bond;

R₂ is alkyl of from 1 to 6 carbon atoms or alkenyl of from 3 to 6 carbon atoms or cycloalkyl of from 3 to 8 carbon atoms;

R₃ is hydroxy, methoxy or ═NOR₅ where R₅ is hydrogen or lower alkyl; and

R₄ is hydrogen, hydroxy or 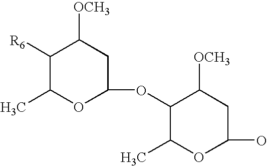
where R₆ is hydroxy, amino, mono- or di-lower alkylamino or lower alkanoylamino.

The preferred compounds are avermectin BIa/BIb (abamectin), 22,23-dihydro avermectin BIa/BIb (ivermectin) and the 4″-acetylamino-5-ketoximino derivative of avermectin BIa/BIb. Both abamectin and ivermectin are approved as broad spectrum antiparasitic agents. The structures of abamectin and ivermectin are as follows: 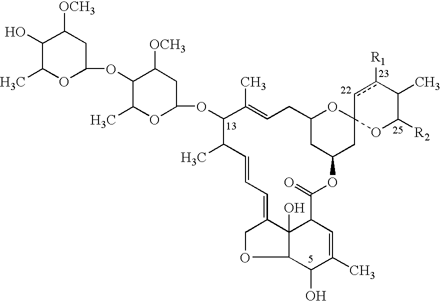
wherein for abamectin the broken line represents a double bond and R₁ is not present and for ivermectin the double bond represents a single bond and R₁ is hydrogen; and R₂ is isopropyl or sec-butyl.

The 4″-acetylamino-5-ketoximino derivatives of avermectin BIa/BIb has the following structural formula: 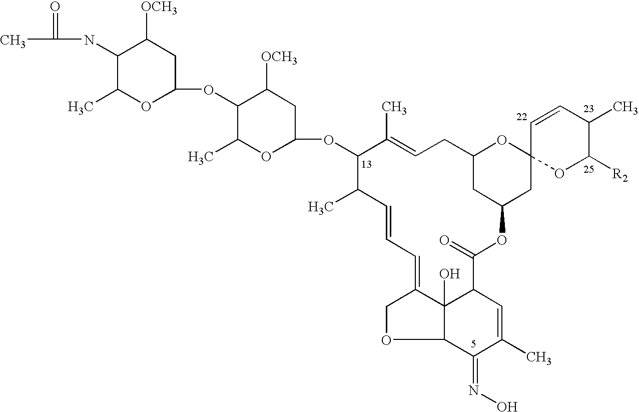
where R₂ is isopropyl or sec-butyl.

The avermectin products are generally prepared as a mixture of at least 80% of the compound where R₂ is sec-butyl and no more than 20% of the compound where R₂ is isopropyl.

Other preferred avermectins, include emamectin, eprinomectin and doramectin. Doramectin is disclosed in U.S. Pat. No. 5,089,490 and EP 214 738. This compound has the following structure: 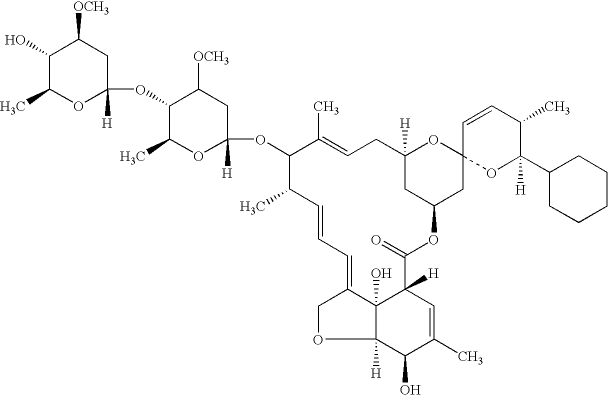
In the present formulations, ivermectin is especially preferred.

A representative structure for a milbemycin is that for milbemycin α₁:

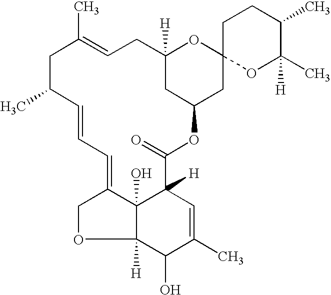

An especially preferred milbemycin is moxidectin, whose structure is as follows: 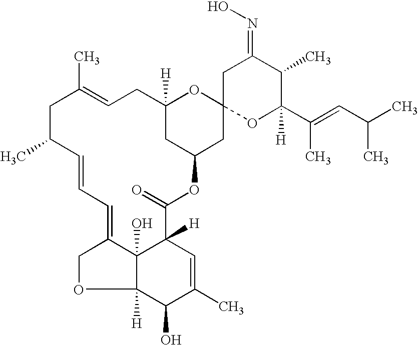
The compound is disclosed in U.S. Pat. No. 5,089,490.

The monosaccharide avermectin derivatives are also preferred especially where an oxime substitution is present on the 5-position of the lactone ring. Such compounds are described, for example, in EP 667,054. Selamectin is an especially preferred compound of this class of derivatives.

This application contemplates all pharmaceutically or veterinary acceptable acid or base salts forms of the anthelmintic compounds, where applicable. The term “acid” contemplates all pharmaceutically or veterinary acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically or veterinary acceptable aliphatic, alicyclic and aromatic carboxylic acids, dicarboxylic acids tricarboxylic acids and fatty acids. Preferred acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, fumaric acid, acetic acid, propionic acid, isopropionic acid, valeric acid, α-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tataric acid and maleic acid. An example of a tricarboxylic acid is citric acid. Fatty acids include all pharmaceutically or veterinary acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

The term “base” contemplates all pharmaceutically or veterinary acceptable inorganic or organic bases. Such bases include, for example, the alkali metal and alkaline earth metal salts, such as the lithium, sodium, potassium, magnesium or calcium salts. Organic bases include the common hydrocarbyl and heterocyclic amine salts, which include, for example, the morpholine and piperidine salts.

The ester and amide derivatives of these compounds, where applicable, are also contemplated. Specific compounds which belong to this class of macrolide antiparasitic agents are well known to the practitioner of this art.

The solvents provided for in the inventive homogeneous pastes or gels are those polar solvent that will dissolve both the first anthelmintic agent and the macrolide anthelmintic compound. These solvents include, for example, glycerol formal, 1-methylpyrrolidone (NMP), dimethyl sulfoxide (DMSO). Glycerol formal exists in two isomeric forms, the α,α′-form and the α,β-form. These forms are reproduced below:

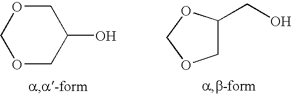

The thickeners contemplated by this invention are well known to a practitioner of this art. Compounds which function as thickeners include, for example, celluloses, starches, natural gums, monothioglycerol, synthetic polymers, such as polymers and copolymers of polyvinylpyrrolidone or (meth)acrylates, etc. Especially preferred thickeners are sodium carboxymethylcellulose (CMC), hydroxypropylcellulose, xanthum gum and hydroxyethyl starch. Thickeners may be present in amounts of from about 3% to about 30%.

Advantageously, the thickener is silica, in particular an anhydrous silica, even more particularly a colloidal anhydrous silica. In an advantageous embodiment, the colloidal anhydrous silica is at a concentration of about 4.5% w/w.

Opacifiers may be added to absorb and/or reflect certain light and/or energy of certain wavelengths and may thus enhance the stability of the formulations. Opacifiers include, for example, zinc oxide or titanium dioxide and may be present in amounts from about 0.5 to 2.5%. Titanium dioxide is especially preferred. These compounds are well known to practitioners of this art.

Additionally, the inventive formulations may contain other inert ingredients such as antioxidants, preservatives, or pH stabilizers. These compounds are well known in the formulation art. Antioxidant such as an alpha tocopheral, ascorbic acid, ascrobyl palmitate, fumaric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. The antioxidants are generally added to the formulation in amounts of from about 0.01 to about 2.0%, based upon total weight of the formulation, with about 0.05 to about 1.0% being especially preferred. Preservatives, such as the parabens (methylparaben and/or propylparaben), are suitably used in the formulation in amounts ranging from about 0.01 to about 2.0%, with about 0.05 to about 1.0% being especially preferred. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Preferred ranges for these compounds include from about 0.01 to about 5%.

Colorants may be added to the inventive formulations. Colorants contemplated by the present invention are those commonly known in the art. Specific colorants include, for example, dyes, an aluminum lake, caramel, colorant based upon iron oxide or a mixture of any of the foregoing. Especially preferred are organic dyes and titanium dioxide. Preferred ranges include from about 0.5% to about 25%.

Compounds which stabilize the pH of the formulation are also contemplates. Again, such compounds are well known to a practitioner in the art as well as how to use these compounds. Buffering systems include, for example, systems selected from the group consisting of acetic acid/acetate, malic acid/malate, citric acid/citrate, tartric acid/tartrate, lactic acid/lactate, phosphoric acid/phosphate, glycine/glycimate, tris, glutamic acid/glutamates, sodium carbonate and sodium carboxymethylcellulose (CMC). Preferred ranges for pH include from about 4 to about 6.5.

The present invention also encompasses flavorings. In an advantageous embodiment, the flavoring may be a mint flavor, fruit flavor, an herb flavor or a spice flavor. Mint flavors include, but are not limited to peppermint, spearmint and wintergreen. Fruit flavors include, but are not limited to, apple, apricot, banana, blackberry, blueberry, cherry, clementine, coconut, cranberry, currant, grape, grapefruit, huckleberry, juniper berry, kiwifruit, lemon, lime, mandarin, melon, orange, papaya, peach, pear, pineapple, plum, pomegranate, raspberry, strawberry, tangerine and watermelon flavors. Herb and/or spice flavors include, but are not limited to, allspice, ancho chile, anise, basil, bay leaf, black pepper, black walnut, caraway seed, cardamom, celery seed, chai-tea, chamomile, chervil, chickory, chipotle chile, chives, cilantro, cinnamon, citrus ginger, clove, coriander, cumin, dill, elderflower, fennel, ginger, ginseng, guarana, hot pepper, jalapeno, jamaica, jasmine, lavender, lemon grass, licorice, mace, marjoram, mint, mustard, nutmeg, oregano, paprika, parsley, poppy seed, pure vanilla, red pepper, rose, rosemary, saffron, sage, savory, sesame seed, tamarind, tarragon, tea, teaberry, thyme, turmeric, vanilla and white pepper. The flavors may be natural or artificial.

In an advantageous embodiment, the flavoring is a peppermint flavor or an apple flavor, advantageously an artificial apple flavor. In another advantageous embodiment, the flavoring is a cinnamon flavor.

The inventive pastes or gels may be administered to warm-blooded animals. Warm-blooded animals include, for example, all ruminants, equines, canines, felines and avians. Especially preferred are birds, cattle, sheep, pigs, dogs, cats, horses and the like. The amount of each of anthelmintic compounds is well known to a practitioner of this art. Preferred amounts of praziquantel include, for example, from about 0.5 mg/kg to about 7.5 mg/kg of animal body weight, with a range of about 0.5 mg/kg to about 2 mg/kg or 2.5 mg/kg of body weight being especially preferred. A most especially preferred amount is about 1.0 mg/kg of animal body weight. Preferred ranges for the anthelmintic macrolide compounds include, for example about 0.01 to about 200 mg/kg of animal body weight, with the ranges of about 0.1 to about 50 mg/kg and from about 1 to about 30 mg/kg being especially preferred.

The inventive oral homogeneous pastes may be prepared, for example, by a process which comprises:

dissolving the at least two different anthelmintic agents, e.g., praziquantel or pyrantel and macrolide anthelmintic compound or compounds, into the solvent; and

adding the thickening agent or agents and stirring until a homogeneous paste is formed. More preferred processes comprise:

dissolving the at least two different anthelmintic agents, e.g., praziquantel or pyrantel, and macrolide anthelmintic compound or compounds, and thickening agent or agents into the solvent and forming a thickened solution;

cooling the thickened solution to a temperature below about 35° C.

adding the viscosity modifier agent and stirring until a homogeneous paste is formed or dissolving the at least two different anthelmintic agents, e.g., praziquantel or pyrantel, and macrolide anthelmintic compound or compounds, the thickening agent or agents and least one compound selected from the group consisting of an antioxidant, a colorant, a pH stabilizer and/or a preservative into the solvent and forming a thickened solution;

cooling the solution to a temperature below about 35° C.; and

adding the viscosity modifying agent or agents and stirring until a homogeneous paste is formed.

A preferred process to prepare the inventive oral veterinary compositions comprises:

dissolving the at least two different anthelmintic agents, e.g., praziquantel or pyrantel, and at least one macrolide anthelmintic compound or compounds and the thickening agent or agents into the solvent and forming a thickened solution;

adding the opacifier to the thickened solution and mixing until the opacifier is evenly dispersed;

cooling the thickened solution with the evenly dispersed opacifier to a temperature below about 35° C.;

adding the viscosity modifier and stirring until the oral veterinary composition is formed.

The inventive oral veterinary formulations may be used to treat a number of ecto- and endoparasite infections. The determining of a treatment protocol for an infection of a specific parasite or parasites would be well within the skill level of a practitioner of the veterinary art. This invention further provides for a method to increase the bioavailability of the at least two different anthelmintic agents in the animal.

The invention will now be further described by way of the following non-limiting examples.

### EXAMPLES

### Example 1

### Oral Veterinary Homogeneous Paste

A better understanding of the present invention and of its many advantages will be had from the following example, given by way of illustration.

An oral veterinary homogeneous paste, which had the following ingredients:

| INGREDIENTS | AMOUNT (% w/w) |
|---|---|
| Praziquantel | 7.75 |
| Ivermectin | 1.55 |
| Butylated hydroxyanisole (BHA) | 0.02 |
| Sunset Yellow (FD&C Yellow No. 6) | 0.04 |
| Titanium dioxide | 2.0 |
| Hydroxypropylcellulose (HPC) | 6.0 |
| Hydrogenated castor oil | 4.0 |
| Stabilized glycerol formal | QS AD 100 |

was prepared by the following process:
1. Add some or all of the stabilized glycerol formal to a mixture followed by the addition of the praziquantel, ivermectin and BHA. The ingredients are mixed until they are dissolved in the stabilized glycerol formal at a temperature above 35° C.
2. Add sunset yellow to the solution and mix until dissolved.
3. Add titanium dioxide to the solution and mix until completely dispersed.
4. Add the remainder of glycerol formal, if necessary.
5. Add HPC to the solution and mix the solution until a homogeneous, viscous solution is obtained.
6. Cool the solution to a temperature below 35° C.
7. Once the solution is cooled to a temperature below 35° C., add the hydrogenated castor oil, while mixing, until all the hydrogenated castor oil is mixed into the solution; the temperature of the solution is maintained below 35° C.
8. Once the hydrogenated castor oil has been added, increase the agitation speed of the mixer while heating the mixture.
9. Mix until the product is a paste.

### Example 2

### Use of Statistically Designed Experiments for Formulation Optimization of a Semisolid

A lower percentage of active was desired in a paste formulation with a resulting increase in the percentage of solvent. Optimization studies of the formulation were undertaken in an effort to eliminate or reduce paste separation and the results are summarized below.

It is believed that the structure of the paste formed from hydrogen bonding between the colloidal silicon dioxide and the polyethylene glycol (see, e.g., Raghavan et al., Langmuir 2000, 16, 7920-7930). The main objective of this example was to test the effect of the following factors on the physical stability (phase separation) of the paste formulation:

(a) amount of colloidal silicon dioxide (Cab-O-Sil) at either 4 or 5%

(b) type of magnesium carbonate (light or heavy)

(c) type of polyethylene glycol (PEG 300 or PEG 400)

(d) shear used during manufacture (high or low shear)

(e) temperature of paste during manufacture (25 C or 45 C)

A stock solution of active in triacetin was prepared. To 15 g of triacetin-drug stock solution, titanium dioxide, magnesium carbonate (light or heavy), and 4% w/w or 5% w/w colloidal silicon dioxide was added using a Lightnin mixer with an appropriate size impeller. The mixer speed was set at either 300 rpm (low shear) or 800 rpm (high shear) and the beaker was set in a circulating water bath maintained at either 25 C or 45 C according to the requirements of the experimental run. As a final step, remaining triacetin and viscosity modifier (PEG 300 or PEG 400) were added to the beaker while mixing so as to make a 50 g paste. Paste was removed from the beaker and approximately 8 grams was centrifuged at 15,000 rpm for 15 minutes and the resulting supernatant liquid was weighed.

TABLE 1
Design of Experiments and their results

| | Colloidal | | Viscosity | | | | | |
|---|---|---|---|---|---|---|---|---|
| Run | SiO₂, | MgCO₃ | Modifier | Shear | Temp. | Weight of liquid, g | | |
| No. | % w/w | type | type | Intensity | (C.) | Tube 1 | Tube 2 | Avg. |
| 1 | 4 | Heavy | PEG400 | Low | 45 | 3.0726 | 2.9635 | 3.018 |
| 2 | 5 | Heavy | PEG300 | Low | 45 | 2.4013 | 2.2292 | 2.315 |
| 3 | 4 | Heavy | PEG400 | High | 25 | 3.1701 | 3.0601 | 3.115 |
| 4 | 5 | Light | PEG300 | Low | 25 | 1.8128 | 1.7567 | 1.785 |
| 5 | 5 | Heavy | PEG300 | High | 25 | 1.7167 | 1.6695 | 1.693 |
| 6 | 5 | Heavy | PEG400 | Low | 25 | 1.6302 | 1.7079 | 1.669 |
| 7 | 5 | Light | PEG400 | High | 25 | 1.8434 | 1.8817 | 1.863 |
| 8 | 4 | Heavy | PEG300 | Low | 25 | 2.8748 | 2.8450 | 2.860 |
| 9 | 5 | Heavy | PEG400 | High | 45 | 1.8680 | 1.8740 | 1.871 |
| 10 | 4 | Light | PEG400 | Low | 25 | 2.9016 | 2.8292 | 2.865 |
| 11 | 5 | Light | PEG300 | High | 45 | 1.7862 | 1.8679 | 1.827 |
| 12 | 4 | Light | PEG300 | High | 25 | 2.7648 | 2.8402 | 2.803 |
| 13 | 4 | Heavy | PEG300 | High | 45 | 2.8487 | 2.6880 | 2.768 |
| 14 | 5 | Light | PEG400 | Low | 45 | 1.9213 | 1.9697 | 1.946 |
| 15 | 4 | Light | PEG400 | High | 45 | 3.0419 | 3.0740 | 3.058 |
| 16 | 4 | Light | PEG300 | Low | 45 | 3.0446 | 2.8130 | 2.929 |

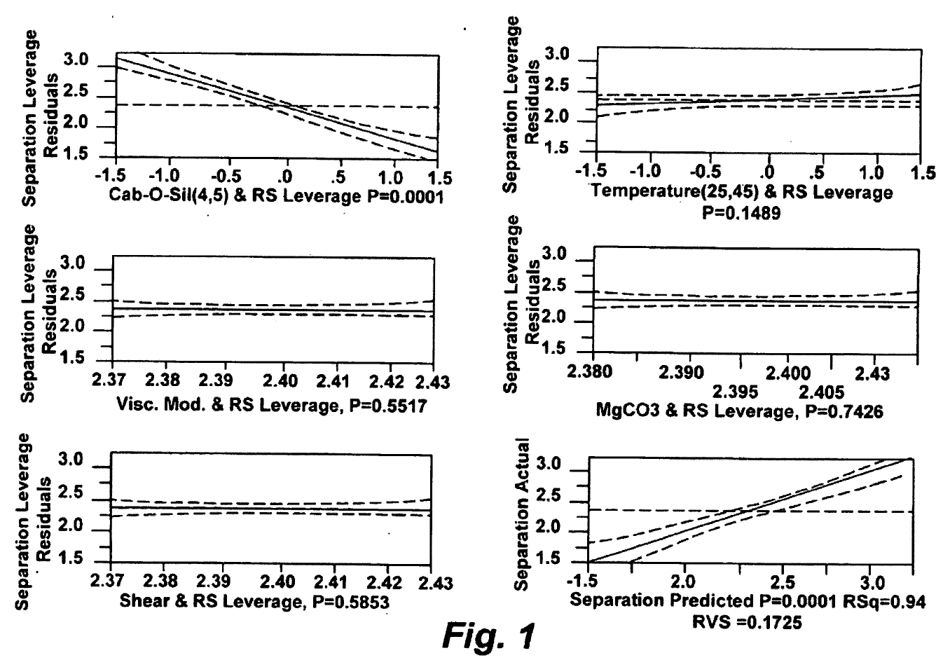

The leverage plots are provided for each of the variable factors as well as the whole model as shown in FIG. 1. The strength of the effect is shown by the slope of the central fit line. The greater the slope (positive or negative), the greater the effect that variable has on the paste separation. The distance from each point to the central fit line is what the error would be if the variable is taken out of the model. Confidence curves on the graph show whether an effect is significant or not. If the 95% confidence curves cross from the horizontal reference line, then the effect is significant; if the curves do not cross, then it is not significant.

From the plots of FIG. 1, it is evident that only the amount of colloidal dioxide in the formulation is very significant and the effect of temperature is marginally significant.

The following table provides the parameters for the variables when the desirability function is maximized. Predicted formulation for the least amount of phase separation is shown in Table 2.

TABLE 2
Predicted formulation

| Variable Factor | Desired Value |
|---|---|
| Colloidal silicon dioxide | 5% w/w |
| MgCO₃ | Light |
| Viscosity Modifier | PEG 400 |
| Shear | Low |
| Temperature | 25 C. |

### Example 3

### Oral Paste for Horses

An oral paste for horses contains firocoxib as active substance, a non-steroidal anti-inflammatory drug (NSAID) that reduces prostaglandin biosynthesis through the selective inhibition of cyclooxygenase-2 (COX-2).

The intended indication in horses is the alleviation of pain and inflammation in animals with ortheoarthritis and reduction of associated lameness. The intended therapeutic dose in horses is 0.1 mg firocoxib/kg bw/day orally for up to 14 consecutive days. The 0.82% oral paste for horses is presented in pre-filled oral syringes containing 7.32 g of oral paste labelled in 100-kg dosing increments. Each syringe contains sufficient product to treat a 600 kg horse.

TABLE 3
Qualitative and Quantitative Composition
of the Medicinal Product

| | | | | Reference to |
|---|---|---|---|---|
| Qualitative | Quantitative | | | analytical |
| Composition | composition | | Function | quality |
| Active Substance | 0.82% | w/w | Active | In-house |
| Firocoxib | | | substance | monograph |
| Excipients | | | | |
| Titanium Dioxide | 0.20% | w/w | Colourant | Ph. Eur. |
| (E171) | | | | |
| Magnesium Carbonate | 2.00% | w/w | Adsorbant | Ph. Eur. |
| (heavy) | 4.50% | w/w | Thickener | Ph. Eur. |
| Colloidal Anhydrous | 0.40% | w/w | Viscosity | Ph. Eur. |
| Silica | | | modifier | |
| Polyethylene Glycol | To 100% | w/w | Vehicle | Ph. Eur. |
| 300 Triacetin | | | | |

As detailed above, early formulations contained active substance concentrations ranging from 0.41-2.05%. The final active substance concentration was chosen with reference to the dose (0.1 mg/kg) and convenience of administration to horses up to 600 kg bodyweight in plastic syringes which are widely used for this dosage form.

The active substance is poorly soluble in water and triacetin was the vehicle of choice because of its ability to solubilise firocoxib and the stability of the resultant solutions under accelerated conditions. Because the active substance is dissolved in the vehicle, particle size and polymorphic properties of the active substance are not relevant to the bioavailability of the formulation. In order to produce a semi solid formulation, several viscosity modifying agents, in combination with colloidal silicon dioxide were investigated. Formulations containing combinations of colloidal silicon dioxide, monoethanolamine and polyethylene glycol 300 (PEG 300) were investigated for viscosity, manageability and penetration values. From experience with previous paste formulations the applicant concludes that a penetration value of between 6-40 mm is ideal in order to ensure that the paste is manageable but does not drip from the syringe barrel when inverted. Good paste viscosity (with penetrometer) was achieved over a wide range of PEG 300 concentrations.

Titanium dioxide was included to improve the appearance of the paste. Following stability studies at 60° C. it was decided that addition of an adsorbent was necessary in order to reduce liquid separation in the syringe on storage. Magnesium carbonate is widely used to adsorb liquids in the tabletting process. Its compatability with the formulation was established and the differences between heavy and light grades were investigated during development. No significant differences in penetration values were observed between batches manufactured using heavy and light grades from the same supplier.

Various development studies were carried out in order to optimise the formulation, to ensure satisfactory penetration value, minimise liquid separation and optimise the concentration of other components. Triacetin, colloidal silicon dioxide, polyethylene glycol 300, titanium dioxide and magnesium carbonate are all monographed in the Ph.Eur. and are generally regarded as non-toxic and non-irritant materials.

Batch instructions for the final pilot scale formulations stated the level of colloidal silicon dioxide to be used as 4.0-4.5%. The amount dispensed for these batches was 4.25%. However during storage of in-process samples in the laboratory it was noted that there was some accumulation of clear liquid in small concavities on the surface of the paste. On testing both the liquid and paste phases were found to contain approximately equal quantities of active substance. Because early development batches demonstrated no differences in physico-chemical properties of paste produced with 4.25% and 4.50% colloidal silicon dioxide, commercial batches will be manufactured with 4.50% colloidal silicon dioxide in order to minimise liquid separation.

Magnesium carbonate is widely used to adsorb liquids at a concentration of 0.5-1.0%. The paste contains 2% of magnesium carbonate, which is already above the typical concentration range. The paste contains 4.5% colloidal silicon dioxide which is typically used at concentrations ranging 2-10% as a suspending and thickening agent in semi-solid preparations. Hence it was decided to increase the concentration of colloidal silicon dioxide rather than magnesium carbonate. Additionally, formulations with 4% magnesium carbonate showed lower penetration values (higher viscosity) as compared to formulations with 2% magnesium carbonate. It is important that a minimum penetration value specification of 6 mm be maintained for proper flow characteristics and there is an increased risk that the penetration value would not meet this specification throughout shelf life of the product if higher levels of magnesium carbonate were utilised.

### Example 4

### A Palatable Oral Gel Dosage Form Containing Eprinomectin for Horses

A clear gel formulation containing eprinomectin was developed as an oral dosage form for horses and ponies for the treatment of internal parasites. The gel formulation can be administered directly from a syringe onto the horse's tongue.

In addition to elegance, an oral gel formula provides improved consistency in delivering the dose as compared to suspensions or paste. Manufacturability is simpler than suspensions or pastes.

TABLE 4
Examples of components and compositions
of an oral gel formultation:

| | Example 1 | Example 2 |
|---|---|---|
| INGREDIENTS | % w/w | % w/w |
| Eprinomectin | 0.050 | 0.050 |
| Sodium Ascorbate | 1.0 | 1.0 |
| Potassium Sorbate | 0.5 | 0.5 |
| Propylene glycol | 20.0 | 0.0 |
| Polyethylene glycol 400 | 0.0 | 20.0 |
| Peppermint Flavor | 0.0 | 1.0 |
| Art. Apple Flavor | 1.5 | 0.0 |
| Sodium CMC | 3.9 | 3.9 |
| Water | 73.0 | 73.5 |
| Total | 100.0 | 100.0 |

The formulation was prepared by first dissolving eprinomectin in propylene glycol or polyethylene glycol. This solution is added to a dispersion of sodium carboxymethylcellulose (Na-CMC) in water. The liquid flavor, sodium ascorbate and potassium sorbate are added and mixed until a clear homogenious system is obtained.

While the examples above were carried out with eprinomectin, other active ingredients would be expected to provide similar results in oral gel formulation. Other gelling agents such as xanthan gum, citrus pectin, sodium alginate, poloxamers, carbomers as well as other cellulose gums can also be utilized in the formulation. In addition to horses and ponies, the oral gel formulation is also suited for use in swine, sheep and cattle.

The invention is further described by the following numbered paragraphs:

1. An oral homogeneous veterinary paste consisting essentially of praziquantel and/or pyrantel and at least one macrolide anthelmintic compound, a solvent, which dissolves both the praziquantel and/or pyrantel and the macrolide anthelmintic compound, and at least one thickening agent.

2. An oral homogeneous veterinary paste consisting essentially of praziquantel and/or pyrantel and at least one macrolide anthelmintic compound, a solvent, which dissolves both the praziquantel and/or pyrantel and the macrolide anthelmintic compound, at least one thickening agent, and at least one viscosity modifier.

3. An oral homogeneous veterinary paste consisting essentially of praziquantel and/or pyrantel and at least one macrolide anthelmintic compound, a solvent, which dissolves both the praziquantel and/or pyrantel and the macrolide anthelmintic compound, at least one thickening agent, a viscosity modifier and at least one compound selected from the group consisting of an antioxidant, a colorant, a pH stabilizer and a preservative.

4. An oral veterinary composition consisting essentially of an oral homogeneous veterinary paste according to any one of paragraphs 1 to 3 and an opacifier.

5. An oral veterinary composition according to paragraph 4, wherein said composition is non-aqueous.

6. An oral veterinary composition according to any one of paragraphs 1 to 3 wherein said composition is non-aqueous.

7. The oral homogeneous paste according to paragraph 1 wherein the macrolide anthelmintic compound is selected from the group consisting of doramectin, abamectin, moxidectin, selamectin, eprinomectin and milbemycin; the solvent is glycerol formal, propylene glycol, 1-methylpyrrolidone, or dimethyl sulfoxide; and the thickening agent is selected from the group consisting of a cellulose, a starch, monothioglycerol, a natural gum, a polymer or copolymer of polyvinylpyrrolidone, and a polymer or copolymer of (meth)acrylate.

8. An oral veterinary composition consisting essentially of an oral homogeneous veterinary paste according to paragraph 7 and an opacifier.

9. The oral homogeneous paste according to paragraph 7 wherein the macrolide anthelmintic compound is ivermectin.

10. The oral veterinary composition according to paragraph 8 wherein the macrolide anthelmintic compound is ivermectin.

11. The oral homogeneous paste according to paragraph 2 wherein the macrolide anthelmintic compound is selected from the group consisting of doramectin, abamectin, moxidectin, selamectin, eprinomectin and milbemycin; the solvent is glycerol formal, propylene glycol, n-methylpyrrolidone, or dimethyl sulfoxide; the thickening agent is selected from the group consisting of a cellulose, a starch, monothioglycerol, polymers or copolymers of polyvinylpyrrolidone, polymers and copolymers of (meth)acrylate, and a natural gum; and the viscosity modifier is selected from the group consisting of vegetable oils, or hydrogenated vegetable oils.

12. The oral homogeneous paste according to paragraph 11 wherein the thickening agent is hydroxypropylcellulose, xanthan gum or hydroxyethyl starch and the viscosity modifier is hydrogenated castor oil, corn oil or olive oil.

13. The oral homogeneous paste according to paragraph 12 wherein the macrolide anthelmintic compound is ivermectin.

14. An oral veterinary composition consisting essentially of an oral homogeneous veterinary paste according to paragraph 11 and an opacifier.

15. The oral veterinary composition according to paragraph 14 wherein the thickening agent is hydroxypropylcellulose, xanthum gum or hydroxyethyl starch and the viscosity modifier is hydrogenated castor oil, corn oil or olive oil and the opacifier is selected from the group consisting of titanium dioxide and zinc oxide.

16. The oral veterinary composition according to paragraph 15 wherein the macrolide anthelmintic compound is ivermectin and the opacifier is titanium dioxide.

17. The oral homogeneous paste according to paragraph 3 wherein

the macrolide anthelmintic compound is selected from the group consisting of doramectin, abamectin, eprinomectin, moxidectin, selamectin and milbemycin;

the solvent is glycerol formal, propylene glycol, n-methylpyrrolidone, or dimethyl sulfoxide; the thickening agent is selected from the group consisting of a cellulose, a starch, monothioglycerol, polymers or copolymers of polyvinylpyrrolidone, polymers or copolymers of (meth)acrylate, and a natural gum;

the viscosity modifier is selected from the group consisting of vegetable oils and hydrogenated vegetable oils.

the antioxidant is selected from the group consisting of alpha tocopherol, ascorbic acid, ascrobyl palmitate, fumaric acid, malic acid, sodium ascorbate, sodium metabisulfate, n-propyl gallate, butylated hydroxy anisole, butylated hydroxy toluene, monothioglycerol;

the colorant is dye, an aluminum lake, caramel, and colorant based upon iron oxide;

the pH stabilizer is a buffering system selected from the group consisting of acetic acid/acetate, malic acid/malate, citric acid/citrate, tartric acid/tartrate, lactic acid/lactate, phosphoric acid/phosphate, glycine/glycimate, tris, glutamic acid/glutamates and sodium carbonate; and

the preservative is a compound selected from the group consisting of benzalkonium chloride, benzethonium chloride, benzoic acid, benzyl alcohol, bronopol, butylparaben, centrimide, chlorhexidine, chlorobutanol, chlorocresol, cresol, ethylparaben, imidurea, methylparaben, propylparaben, phenol, phenoxyethanol, phenylethyl, alcohol, phenylmercuric acetate, pheylmecuric borate, phenylmercuric nitrate, potassium sorbate, sodium benzoate, sodium propionate, sorbic acid, and thimerosal.

18. The oral homogeneous paste according to paragraph 17 wherein the macrolide anthelmintic compound is ivermectin.

19. The oral homogeneous veterinary paste according to paragraph 3, wherein the macrolide anthelmintic compound is ivermectin, the solvent is glycerol formal, the thickener is hydroxypropylcellulose, the viscosity modifier is hydrogenated castor oil, the colorant is an organic dye, and the preservative is selected from the group consisting of butylated hydroxytoluene or butylated hydroxy anisole.

20. The oral homogeneous veterinary paste according to paragraph 19 wherein the dye is an organic dye which is sunset yellow.

21. The oral veterinary composition consisting essentially of a an oral homogeneous veterinary paste according to paragraph 17 and an opacifier.

22. The oral veterinary composition consisting essentially of an oral homogeneous veterinary paste according to paragraph 19 and an opacifier.

23. The oral veterinary composition according to paragraph 20 wherein the dye is an organic dye which is sunset yellow

24. The oral veterinary composition according to paragraph 4 which is

| | | |
|---|---|---|
| praziquantel | 7.75% | w/w |
| ivermectin | 1.55% | w/w |
| butylated hydroxyanisole | 0.02% | w/w |
| sunset yellow (FD&C Yellow No. 6) | 0.04% | w/w |
| titanium dioxide | 2.0% | w/w |
| hydroxypropylcellulose | 6.0% | w/w |
| hydrogenated castor oil | 4.0% | w/w |

| | |
|---|---|
| stabilized glycerol formal | amount to make 100%. |

25. The oral homogeneous paste according to any one of paragraph 1 to 3, wherein the first anthelmintic agent is praziquantel.

26. The oral homogeneous paste according to paragraph 4, wherein the first anthelmintic agent is praziquantel.

27. A process for preparing an oral homogeneous veterinary paste according to paragraph 1 which comprises

dissolving the praziquantel and/or pyrantel and macrolide anthelmintic compound or compounds into the solvent; and

adding the thickening agent or agents and stirring until a homogeneous paste is formed.

28. A process for preparing an oral homogeneous veterinary paste according to paragraph 2 which comprises

dissolving the praziquantel and/or pyrantel and macrolide anthelmintic compound or compounds and thickening agent or agents into the solvent and forming a thickened solution;

cooling the thickened solution to a temperature below about 35° C.; and

adding the viscosity modifier agent or agents and stirring until a homogeneous paste is formed.

29. A process for preparing an oral homogeneous veterinary paste according to paragraph 3 which comprises:

dissolving the praziquantel and/or pyrantel and macrolide anthelmintic compound or compounds, the thickening agent or agents and least one compound selected from the group consisting of an antioxidant, a colorant, a pH stabilizer and/or a preservative into the solvent and forming a thickened solution; and

cooling the thickened solution to a temperature below about 35 C; and

adding the viscosity modifying agent or agents and stirring until a homogeneous paste is formed.

30. A process for preparing an oral veterinary composition according to paragraph 4 which comprises:

dissolving the parzequantel and at least one macrolide anthelmintic compound or compounds and the thickening agent or agents into the solvent and forming a thickened solution;

adding the opacifier to the thickened solution and mixing until the opacifier is evenly dispersed;

cooling the thickened solution with the evenly dispersed opacifier to a temperature below about 35° C.;

adding the viscosity modifier and stirring until the oral veterinary formulation is formed.

31. A method for increasing the bioavailability of praziquantel and at least one macrolide anthelmintic compound in a warm-blooded animal which comprises administering the oral homogeneous veterinary paste according to any one paragraphs 1 to 3 said warm-blooded animal or bird.

32. A method for increasing the bioavailability of praziquantel and at least one macrolide anthelmintic compound in a warm-blooded animal or bird which comprises administering the oral veterinary composition according to paragraph 4 to said warm-blooded animal or bird.

33. An oral veterinary paste consisting essentially of dissolved praziquantel and dissolved ivermectin.

34. The oral veterinary paste of paragraph 33 wherein the praziquantel and ivermectin are both dissolved in glycerol formal.

35. The oral veterinary paste of paragraph 34 further consisting essentially of praziquantel and ivermectin dissolved in glycerol formal and a cellulose.

36. The oral veterinary paste of paragraph 35 further consisting essentially of hydrogenated caster oil.

37. The oral veterinary paste of paragraph 35, wherein the cellulose is hydroxypropyl cellulose.

38. The oral veterinary paste of paragraph 36 further consisting essentially of antioxidant, colorant, titanium dioxide.

39. The oral veterinary paste of paragraph 38 wherein the cellulose is hydroxypropyl cellulose, the antioxidant is butylated hydroxyanisole and the colorant is sunset yellow (FD&C Yellow No. 6).

40. The oral veterinary paste of paragraph 33 further consisting essentially of a cellulose, hydrogenated castor oil, and glycerol formal.

41. The oral veterinary paste according to paragraph 40 wherein the cellulose is hydroxypropylcellulose.

42. The oral veterinary paste of paragraph 33 further consisting essentially of a cellulose, hydrogenated castor oil, glycerol formal and one or more compounds selected from the group consisting of an antioxidant, an opacifier and a colorant.

43. The oral veterinary paste according to paragraph 42 wherein the cellulose is hydroxypropylcellulose.

44. A method for increasing the bioavailability of praziquantel and a macrolide anthelmintic compound in a warm-blooded animal which comprises administering the oral veterinary paste according to paragraph 33 to said warm-blooded animal.

45. The method of paragraph 44 wherein the warm-blooded animal is bird, cattle, sheep, pig, dog, cat or horse.

46. The method of paragraph 45 wherein the warm-blooded animal is a bird.

47. The method of paragraph 45 wherein the warm-blooded animal is a horse.

50. An oral veterinary paste consisting essentially of praziquantel, ivermectin, antioxidant, colorant, titanium dioxide, a cellulose, hydrogenated castor oil, and glycerol formal.

51. The oral veterinary paste according to paragraph 50 wherein the cellulose is hydroxypropylcellulose.

52. The oral veterinary paste of paragraph 50, wherein the cellulose is hydroxypropylcellulose and the glycerol formal is stabilized glycerol formal.

53. The oral veterinary paste of paragraph 50 which is produced by the process comprising:

(a) dissolving praziquantel, ivermectin, colorant, titanium dioxide, antioxidant and cellulose into glycerol formal to form a thickened solution;

(b) cooling the thickenened solution to a temperature below about 35° C.;

(c) adding hydrogenated castor oil to the thickened solution and stirring until a homogenous paste is formed,

wherein the cellulose is hydroxypropyl cellulose and wherein the antioxidant is butylated hydroxyanisole, the opacifier is titanium dioxide and the colorant is sunset yellow (FD&C Yellow No. 6).

54. The oral veterinary paste according to paragraph 50, which is

| | | |
|---|---|---|
| praziquantel | 7.75 | (% w/w) |
| ivermectin | 1.55 | (% w/w) |
| butylated hydroxyanisole | 0.02 | (% w/w) |
| sunset yellow (FD&C Yellow No. 6) | 0.04 | (% w/w) |
| titanium dioxide | 2.0 | (% w/w) |
| hydroxypropylcellulose | 6.0 | (% w/w) |
| hydrogenated castor oil | 4.0 | (% w/w) |

| | |
|---|---|
| glycerol formal | QS AD 100. |

55. An oral veterinary paste comprising:

| | | |
|---|---|---|
| praziquantel | 7.75 | (% w/w) |
| ivermectin | 1.55 | (% w/w) |
| hydroxypropylcellulose | 6.0 | (% w/w) |
| hydrogenated castor oil | 4.0 | (% w/w) |

| | |
|---|---|
| glycerol formal | QS AD 100 |

and optionally, one or more compounds selected from the group consisting of an antioxidant, an opacifier and a colorant.

56. The oral veterinary paste of paragraph 55, wherein the antioxidant is butylated hydroxyanisole, the opacifier is titanium dioxide and the colorant is sunset yellow (FD&C Yellow No. 6).

57. A method for increasing the bioavailability of praziquantel and a macrolide anthelmintic compound in a warm-blooded animal which comprises administering the oral veterinary paste according to paragraph 50 to said warm-blooded animal.

58. The method of paragraph 57 wherein the warm-blooded animal is bird, cattle, sheep, pig, dog, cat or horse.

59. The method of paragraph 58 wherein the warm-blooded animal is a bird.

60. The method of paragraph 58 wherein the warm-blooded animal is a horse.

61. A method for preparing a pharmaceutical or veterinary paste formulation comprising:

dissolving or dispersing a therapeutic agent into a carrier,

adding fumed silica and an absorbent to the carrier containing the dissolved therapeutic agent,

mixing the fumed silica, absorbent and carrier containing the dissolved therapeutic agent at low shear,

maintaining the temperature at about 25 C until the silica and absorbent is dispersed in the carrier and

adding a viscosity modifier to the intermediate with mixing to produce a uniform pharmaceutical or veterinary paste formulation.

62. The method of paragraph 61 wherein the mixing at low shear is at 300 rpm.

63. The method of paragraph 61 wherein the fumed silica is a colloidal silicon dioxide.

64. The method of paragraph 63 wherein the colloidal silicon dioxide is at a final concentration of 5% w/w in the pharmaceutical or veterinary paste formulation.

65. The method of paragraph 61 wherein the absorbent is magnesium carbonate.

66. The method of paragraph 65 wherein the magnesium carbonate is a light magnesium carbonate.

67. The method of paragraph 61 wherein the viscosity modifier is PEG 400.

68. The method of paragraph 61 wherein

the mixing at low shear is at 300 rpm,

the fumed silica is a colloidal silicon dioxide,

the absorbent is magnesium carbonate and

the viscosity modifier is PEG 400.

69. The method of paragraph 68 wherein

the colloidal silicon dioxide is at a final concentration of 5% w/w in the pharmaceutical or veterinary paste formulation.

70. The method of paragraph 68 wherein

the magnesium carbonate is a light magnesium carbonate.

71. The method of paragraph 68 wherein

the colloidal silicon dioxide is at a final concentration of 5% w/w in the pharmaceutical or veterinary paste formulation and

the magnesium carbonate is a light magnesium carbonate.

72. A pharmaceutical or veterinary paste formulation comprising:

(a) an effective amount of a therapeutic agent,

(b) a fumed silica, wherein the fumed silica is 5% w/w colloidal silicon dioxide,

(c) a viscosity modifier, wherein the viscosity modifier is PEG 400

(d) an absorbent, wherein the absorbent is magnesium carbonate.

73. The pharmaceutical or veterinary paste of paragraph 72, wherein the magnesium carbonate is a light magnesium carbonate.

74. The pharmaceutical or veterinary paste of paragraph 72 wherein the therapeutic agent is selected from the group consisting of avermectins, milbemycins, nordulisporic acid and its derivatives, estrogens, progestins, androgens, substituted pyridyl methyl derivatives, phenylpyrazoles, COX-2 inhibitors or 2-(2-benzimidazolyl)-pyrimidine derivatives.

75. The pharmaceutical or veterinary paste of paragraph 72 wherein the therapeutic agent comprises praziquantel and ivermectin.

76. The pharmaceutical or veterinary paste of paragraph 75 wherein the therapeutic agent comprises dissolved praziquantel and dissolved ivermectin.

77. The pharmaceutical or veterinary paste of paragraph 72 further comprising a carrier.

78. The pharmaceutical or veterinary paste of paragraph 77 wherein the carrier is a triacetin, a monoglyceride, a diglyceride, or a triglyceride.

79. The pharmaceutical or veterinary paste of paragraph 78 wherein the carrier is a triacetin.

80. The pharmaceutical or veterinary paste of paragraph 72 further comprising a colorant, stabilizer, surfactant or preservative.

81. A pharmaceutical or veterinary paste formulation comprising:

(a) an effective amount of a therapeutic agent, wherein the therapeutic agent is a COX-2 inhibitor,

(b) a colorant, wherein the colorant is titanium dioxide,

(c) an absorbent, wherein the absorbent is magnesium carbonate,

(d) a thickener, wherein the thickener is colloidal anhydrous silica,

(e) a viscosity modifier, wherein the viscosity modifier is PEG 300.

82. The pharmaceutical or veterinary paste of paragraph 81, wherein the COX-2 inhibitor is firocoxib.

83. The pharmaceutical or veterinary paste of paragraph 81 or 82, wherein the titanium dioxide is an E171 titanium dioxide.

84. The pharmaceutical or veterinary paste of any one of paragraphs 81 to 83, wherein the magnesium carbonate is a heavy magnesium carbonate.

85. The pharmaceutical or veterinary paste of any one of paragraphs 81 to 84 further comprising a carrier.

86. The pharmaceutical or veterinary paste of paragraph 85 wherein the carrier is a triacetin.

87. The pharmaceutical or veterinary paste of any one of paragraphs 81 to 86, further comprising a colorant, stabilizer, surfactant or preservative.

88. A pharmaceutical or veterinary paste formulation comprising:

(a) an effective amount of a therapeutic agent, wherein the therapeutic agent is a firocoxib,

(b) a colorant, wherein the colorant is titanium dioxide,

(c) an absorbent, wherein the absorbent is magnesium carbonate,

(d) a thickener, wherein the thickener is colloidal anhydrous silica,

(e) a viscosity modifier, wherein the viscosity modifier is PEG 300,

(f) a carrier, wherein the carrier is a triacetin.

89. The pharmaceutical or veterinary paste formulation of paragraph 88, wherein the firocoxib is about 0.82% w/w.

90. The pharmaceutical or veterinary paste formulation of paragraph 88 or 89, wherein the titanium dioxide is an E171 titanium dioxide.

91. The pharmaceutical or veterinary paste formulation of any one of paragraphs 88 to 90, wherein the titanium dioxide is about 0.2% w/w.

92. The pharmaceutical or veterinary paste formulation of any one of paragraphs 88 to 91, wherein the magnesium carbonate is a heavy magnesium carbonate.

93. The pharmaceutical or veterinary paste formulation of any one of paragraphs 88 to 92, wherein the magnesium carbonate is about 2% w/w.

94. The pharmaceutical or veterinary paste formulation of any one of paragraphs 88 to 93, wherein the colloidal anhydrous silica is about 4.5% w/w.

95. The pharmaceutical or veterinary paste formulation of any one of paragraphs 88 to 94, wherein the PEG 300 is about 0.4% w/w.

96. The pharmaceutical or veterinary paste formulation of any one of paragraphs 88 to 95, wherein the triacetin is about 92% w/w.

97. A pharmaceutical or veterinary paste formulation comprising:

(a) an effective amount of a therapeutic agent, wherein the therapeutic agent is about 0.82% w/w firocoxib,

(b) a colorant, wherein the colorant is about 0.2% w/w titanium dioxide,

(c) an absorbent, wherein the absorbent is about 2% w/w magnesium carbonate,

(d) a thickener, wherein the thickener is about 4.5% w/w colloidal anhydrous silica,

(e) a viscosity modifier, wherein the viscosity modifier is 0.4% w/w PEG 300,

(f) a carrier, wherein the carrier is about 92% w/w triacetin.

98. A pharmaceutical or veterinary gel formulation comprising:

(a) an effective amount of a therapeutic agent, wherein the therapeutic agent is an avermectin,

(b) an antioxidant, wherein the antioxidant is sodium ascorbate,

(c) a preservative, wherein the preservative is potassium sorbate,

(d) a flavoring, wherein the flavoring is a peppermint or apple flavor and

(e) a buffering system or thickener, wherein the buffering system or thickener is sodium carboxymethylcellulose.

99. The pharmaceutical or veterinary gel formulation of paragraph 98 further comprising a solvent, wherein the solvent is propylene glycol.

100. The pharmaceutical or veterinary gel formulation of paragraph 98 further comprising a viscosity modifier, wherein the viscosity modifier is polyethylene glycol 400.

101. The pharmaceutical or veterinary gel formulation of any one of paragraphs 98 to 100, wherein the avermectin is eprinomectin.

102. The pharmaceutical or veterinary gel formulation of any one of paragraphs 98 to 101, wherein the flavoring is a peppermint flavor.

103. The pharmaceutical or veterinary gel formulation of any one of paragraphs 98 to 101, wherein the flavoring is an apple flavor.

104. The pharmaceutical or veterinary gel formulation of paragraph 103, wherein the flavoring is an artificial apple flavor.

105. A pharmaceutical or veterinary gel formulation comprising:

(a) an effective amount of a therapeutic agent, wherein the therapeutic agent is eprinomectin,

(b) an antioxidant, wherein the antioxidant is sodium ascorbate,

(c) a preservative, wherein the preservative is potassium sorbate,

(d) a flavoring, wherein the flavoring is a peppermint or apple flavor and

(e) a buffering system or thickener, wherein the buffering system or thickener is sodium carboxymethylcellulose.

106. The pharmaceutical or veterinary gel formulation of paragraph 105 further comprising a solvent, wherein the solvent is propylene glycol.

107. The pharmaceutical or veterinary gel formulation of paragraph 105 further comprising a viscosity modifier, wherein the viscosity modifier is polyethylene glycol.

108. The pharmaceutical or veterinary gel formulation of any one of paragraphs 105 to 107, wherein the eprinomectin is about 0.050% w/w.

109. The pharmaceutical or veterinary gel formulation of any one of paragraphs 105 to 108, wherein the sodium ascorbate is about 1.0% w/w.

110. The pharmaceutical or veterinary gel formulation of any one of paragraphs 105 to 109, wherein the potassium sorbate is about 0.5% w/w.

111. The pharmaceutical or veterinary gel formulation of any one of paragraphs 106 and 108 to 110, wherein the propylene glycol is about 20.0% w/w.

112. The pharmaceutical or veterinary gel formulation of any one of paragraphs 107 to 110, wherein the polyethylene glycol is about 20.0% w/w.

113. The pharmaceutical or veterinary gel formulation of any one of paragraphs 105 to 112, wherein the peppermint flavor is about 1.0% w/w.

114. The pharmaceutical or veterinary gel formulation of any one of paragraphs 105 to 112, wherein the apple flavor is about 1.5% w/w.

115. The pharmaceutical or veterinary gel formulation of any one of paragraphs 105 to 114, wherein the sodium carboxymethylcellulose is about 3.9% w/w.

116. A pharmaceutical or veterinary gel formulation comprising:

(a) an effective amount of a therapeutic agent, wherein the therapeutic agent is about 0.050% w/w eprinomectin,

(b) an antioxidant, wherein the antioxidant is about 1.0% w/w sodium ascorbate,

(c) a preservative, wherein the preservative is about 0.5% w/w potassium sorbate,

(d) a flavoring, wherein the flavoring is about 1.0% w/w peppermint or about 1.5% artificial apple flavor and

(e) a buffering system or thickener, wherein the buffering system or thickener is about 3.9% w/w sodium carboxymethylcellulose.

117. The pharmaceutical or veterinary gel formulation of paragraph 116 further comprising a solvent, wherein the solvent is about 20.0% w/w propylene glycol.

118. The pharmaceutical or veterinary gel formulation of paragraph 116 further comprising a viscosity modifier, wherein the viscosity modifier is about 20.0% polyethylene glycol 400.

119. A pharmaceutical or veterinary gel formulation comprising:

(a) an effective amount of a therapeutic agent, wherein the therapeutic agent is ivermectin,

(b) an antioxidant, wherein the antioxidant is sodium ascorbate,

(c) a preservative, wherein the preservative is potassium sorbate,

(d) a flavoring, wherein the flavoring is a peppermint or apple flavor and

(e) a buffering system or thickener, wherein the buffering system or thickener is sodium carboxymethylcellulose.

120. The pharmaceutical or veterinary gel formulation of paragraph 119 wherein the therapeutic agent further comprises praziquantel.

121. A pharmaceutical or veterinary gel formulation comprising:

(a) an effective amount of a therapeutic agent, wherein the therapeutic agent is a proton pump inhibitor,

(b) an antioxidant, wherein the antioxidant is sodium ascorbate,

(c) a preservative, wherein the preservative is potassium sorbate,

(d) a flavoring, wherein the flavoring is a peppermint or apple flavor and

(e) a buffering system or thickener, wherein the buffering system or thickener is sodium carboxymethylcellulose.

122. The pharmaceutical or veterinary gel formulation of paragraph 121 wherein the proton pump inhibitor is omeprazole.

123. A pharmaceutical or veterinary gel formulation comprising:

(a) an effective amount of a therapeutic agent, wherein the therapeutic agent is a COX-2 inhibitor,

(b) an antioxidant, wherein the antioxidant is sodium ascorbate,

(c) a preservative, wherein the preservative is potassium sorbate,

(d) a flavoring, wherein the flavoring is a peppermint or apple flavor and

(e) a buffering system or thickener, wherein the buffering system or thickener is sodium carboxymethylcellulose.

124. The pharmaceutical or veterinary gel formulation of paragraph 123 wherein the COX-2 inhibitor is 3-(cyclopropylmethoxy)-5,5-dimethyl-4-[4-(methylsulfonyl)phenyl]-2(5H)-furanone.

125. The pharmaceutical or veterinary gel formulations of any one of paragraphs 119 to 124 further comprising a solvent, wherein the solvent is propylene glycol.

126. The pharmaceutical or veterinary gel formulation of any one of paragraphs 119 to 125 further comprising a viscosity modifier, wherein the viscosity modifier is polyethylene glycol 400.

128. The pharmaceutical or veterinary gel formulation of any one of paragraphs 119 to 127, wherein the flavoring is a peppermint flavor.

129. The pharmaceutical or veterinary gel formulation of any one of paragraphs 119 to 127, wherein the flavoring is an apple flavor.

130. The pharmaceutical or veterinary gel formulation of paragraph 129, wherein the flavoring is an artificial apple flavor.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A pharmaceutical or veterinary paste formulation comprising:
(a) an effective amount of a therapeutic agent, wherein the therapeutic agent is a COX-2 inhibitor,
(b) a colorant, wherein the colorant is titanium dioxide,
(c) an absorbent, wherein the absorbent is magnesium carbonate,
(d) a thickener, wherein the thickener is colloidal anhydrous silica,
(e) a viscosity modifier, wherein the viscosity modifier is PEG 300.

2. The pharmaceutical or veterinary paste of claim 1,
wherein the COX-2 inhibitor is firocoxib or
wherein the titanium dioxide is an E171 titanium dioxide or
wherein the magnesium carbonate is a heavy magnesium carbonate.

3. The pharmaceutical or veterinary paste of claim 1 further comprising a carrier or the pharmaceutical or veterinary paste of claim 1 further comprising a carrier, wherein the carrier is a triacetin.

4. The pharmaceutical or veterinary paste of claim 1, further comprising a colorant, stabilizer, surfactant or preservative.

5. A pharmaceutical or veterinary paste formulation of claim 1, wherein the therapeutic agent is a firocoxib and further comprising a carrier, wherein the carrier is a triacetin.

6. The pharmaceutical or veterinary paste formulation of claim 5,
wherein the firocoxib is about 0.82% w/w or
wherein the titanium dioxide is an E171 titanium dioxide or
wherein the titanium dioxide is about 0.2% w/w or
wherein the magnesium carbonate is a heavy magnesium carbonate or
wherein the magnesium carbonate is about 2% w/w or
wherein the colloidal anhydrous silica is about 4.5% w/w or
wherein the PEG 300 is about 0.4% w/w or
wherein the triacetin is about 92% w/w.

7. A pharmaceutical or veterinary paste formulation of claim 5 wherein:
(a) the therapeutic agent is about 0.82% w/w firocoxib,
(b) the colorant is about 0.2% w/w titanium dioxide,
(c) the absorbent is about 2% w/w magnesium carbonate,
(d) the thickener is about 4.5% w/w colloidal anhydrous silica,
(e) the viscosity modifier is 0.4% w/w PEG 300,
(f) the carrier is about 92% w/w triacetin.

8. A pharmaceutical or veterinary gel formulation comprising:
(a) an effective amount of a therapeutic agent, wherein the therapeutic agent is an avermectin,
(b) an antioxidant, wherein the antioxidant is sodium ascorbate,
(c) a preservative, wherein the preservative is potassium sorbate,
(d) a flavoring, wherein the flavoring is a peppermint or apple flavor and
(e) a buffering system or thickener, wherein the buffering system or thickener is sodium carboxymethylcellulose.

9. The pharmaceutical or veterinary gel formulation of claim 8, wherein the avermectin is eprinomectin.

10. The pharmaceutical or veterinary gel formulation of claim 8 further comprising a solvent, wherein the solvent is propylene glycol or
further comprising a solvent, wherein the solvent is propylene glycol and wherein the avermectin is eprinomectin or
further comprising a viscosity modifier, wherein the viscosity modifier is polyethylene glycol 400 or
further comprising a viscosity modifier, wherein the viscosity modifier is polyethylene glycol 400 and wherein the avermectin is eprinomectin.

11. The pharmaceutical or veterinary gel formulation of claim 8, wherein the flavoring is a peppermint flavor or
further comprising a solvent, wherein the solvent is propylene glycol and wherein the flavoring is a peppermint flavor or
further comprising a solvent, wherein the solvent is propylene glycol, wherein the avermectin is eprinomectin and wherein the flavoring is a peppermint flavor or
further comprising a viscosity modifier, wherein the viscosity modifier is polyethylene glycol 400 and wherein the flavoring is a peppermint flavor or
further comprising a viscosity modifier, wherein the viscosity modifier is polyethylene glycol 400, wherein the avermectin is eprinomectin and wherein the flavoring is a peppermint flavor or
wherein the flavoring is an apple flavor or
further comprising a solvent, wherein the solvent is propylene glycol and wherein the flavoring is an apple flavor or
further comprising a solvent, wherein the solvent is propylene glycol, wherein the avermectin is eprinomectin and wherein the flavoring is an apple flavor or
further comprising a viscosity modifier, wherein the viscosity modifier is polyethylene glycol 400 and wherein the flavoring is an apple flavor or
further comprising a viscosity modifier, wherein the viscosity modifier is polyethylene glycol 400, wherein the avermectin is eprinomectin and wherein the flavoring is an apple flavor.

12. A pharmaceutical or veterinary gel formulation comprising:
(a) an effective amount of a therapeutic agent,
(b) an antioxidant, wherein the antioxidant is sodium ascorbate,
(c) a preservative, wherein the preservative is potassium sorbate,
(d) a flavoring, wherein the flavoring is a peppermint or apple flavor and
(e) a buffering system or thickener, wherein the buffering system or thickener is sodium carboxymethylcellulose.

13. The pharmaceutical or veterinary gel formulation of claim 12, wherein the therapeutic agent is eprinomectin.

14. The pharmaceutical or veterinary gel formulation of claim 12 further comprising a solvent, wherein the solvent is propylene glycol or further comprising a viscosity modifier, wherein the viscosity modifier is polyethylene glycol.

15. The pharmaceutical or veterinary gel formulation of claim 13, wherein the eprinomectin is about 0.050% w/w or
wherein the sodium ascorbate is about 1.0% w/w or
wherein the potassium sorbate is about 0.5% w/w or
further comprising a solvent, wherein the solvent is propylene glycol and wherein the propylene glycol is about 20.0% w/w or
further comprising a viscosity modifier, wherein the viscosity modifier is polyethylene glycol and wherein the polyethylene glycol is about 20.0% w/w or
wherein the peppermint flavor is about 1.0% w/w or
wherein the apple flavor is about 1.5% w/w or
wherein the sodium carboxymethylcellulose is about 3.9% w/w.

16. A pharmaceutical or veterinary gel formulation of claim 13 comprising:
(a) about 0.050% w/w eprinomectin,
(b) about 1.0% w/w sodium ascorbate,
(c) about 0.5% w/w potassium sorbate,
(d) about 1.0% w/w peppermint or about 1.5% artificial apple flavor and
(e) about 3.9% w/w sodium carboxymethylcellulose.

17. The pharmaceutical or veterinary gel formulation of claim 16 further comprising about 20.0% w/w propylene glycol or
about 20.0% polyethylene glycol 400.

18. The pharmaceutical or veterinary gel formulation of claim 12, wherein the therapeutic agent is ivermectin or a combination of ivermectin and praziquantel.

19. The pharmaceutical or veterinary gel formulation of claim 12, wherein the therapeutic agent is a COX-2 inhibitor, advantageously 3-(cyclopropylmethoxy)-5,5-dimethyl-4-[4-(methylsulfonyl)phenyl]-2(5H)-furanone.

20. The pharmaceutical or veterinary gel formulation of claim 12, wherein the therapeutic agent is a proton pump inhibitor, advantageously omeprazole.
